(19) United States
(12) Patent Application Publication
(10) Pub. No.: US 2025/0082741 A1
(43) Pub. Date: Mar. 13, 2025

(54) Listeria Variants and Methods of Use Thereof

(71) Applicant: The Regents of the University of California, Oakland, CA (US)

(72) Inventors: Daniel A. Portnoy, Albany, CA (US); Rafael Jose Rivera-Lugo, Albany, CA (US)

(21) Appl. No.: 18/728,813

(22) PCT Filed: Jan. 26, 2023
(86) PCT No.: PCT/US2023/011628

§ 371 Date: Jul. 12, 2024

Related U.S. Application Data
(60) Provisional application No. 63/304,235, filed on Jan. 28, 2022.

(51) Int. Cl. A61K 39/02; A61K 35/74; A61K 39/00; A61P 37/04; C07K 16/28; C12N 1/20

(52) U.S. Cl. CPC A61K 39/02; A61K 35/74; A61P 37/04; C07K 16/2809; C12N 1/20; A61K 2039/522; A61K 2039/523; A61K 2039/572

(57) ABSTRACT

The present disclosure provides variant Listeria bacteria that lack functional ribC and ribF genes. Also provided are methods of making and using the variant Listeria bacteria, e.g., as vectors, vaccines, and therapeutics. The present disclosure provides variant Listeria bacteria that provide for production of a Mucosal-Associated Invariant T (MAIT) cell ligand in vivo. Also provided are methods of making and using the variant Listeria bacteria, e.g., to stimulate and expand the number of MAIT cells in an individual.

### CROSS-REFERENCE

This application claims the benefit of U.S. Provisional Patent Application No. 63/304,235, filed Jan. 28, 2022, which application is incorporated herein by reference in its entirety.

### STATEMENT REGARDING FEDERALLY SPONSORED RESEARCH

This invention was made with government support under Grant No. AI027655 and Grant No. AI063302 awarded by the National Institutes of Health. The government has certain rights in the invention.

### INTRODUCTION

Listeria monocytogenes is a Gram-positive, food-borne human and animal pathogen responsible for serious infections in immunocompromised individuals and pregnant women. Severe L. monocytogenes infections in humans are characterized by meningitis, meningoencephalitis, septicemia, and fetal death. L. monocytogenes is ubiquitous in nature and, in addition, can be isolated from a wide variety of warm-blooded animals.

L. monocytogenes elicits a predominantly cellular immune response when inoculated into an animal. As such, L. monocytogenes has been used widely as an experimental model to study many aspects of infection and immunity. Importantly, infection of mice with sublethal doses of L. monocytogenes results in the induction of long-lived cell-mediated immunity (CMI). In preclinical studies, attenuated strains of L. monocytogenes have shown tremendous potential as recombinant vaccine vectors. Attenuated recombinant strains have shown clinical efficacy as therapeutic vaccines for cancer immunotherapy.

Many known prokaryotic organisms depend on a single bifunctional enzyme, encoded by the ribC (or ribF, depending on the organism) gene and named riboflavin kinase/FAD synthetase (FADS), to convert Riboflavin (RF), first into flavin mononucleotide (FMN) and then into flavin adenine dinucleotide (FAD). Listeria has two separate enzymes encoded by the genes ribC and ribF.

### SUMMARY

The present disclosure provides variant Listeria bacteria that lack functional ribC and ribF genes. Also provided are methods of making and using the variant Listeria bacteria, e.g., as vectors, vaccines, and therapeutics. The present disclosure provides variant Listeria bacteria that provide for production of a Mucosal-Associated Invariant T (MAIT) cell ligand in vivo. Also provided are methods of making and using the variant Listeria bacteria, e.g., to stimulate and expand the number of MAIT cells in an individual.

### BRIEF DESCRIPTION OF THE DRAWINGS

FIG. 1A-1D. FIG. 1A depicts the structures of riboflavin, FMN, and FAD. FIG. 1B depicts broth growth curves of L. monocytogenes strains grown in nutrient-rich media. FIG. 1C-1D depict colony-forming units (CFUs) of L. monocytogenes strains in the spleens (C) and livers (D) of mice 48 hours after being infected intravenously with 1×10⁵ CFUs of the respective strains.

FIG. 2A-2B. FIG. 2A depicts broth growth curves of L. monocytogenes strains grown in chemically defined media lacking flavins. FIG. 2B depicts an image of the media supernatant of wild-type (left) and the ΔribU+ribDEAHT strain (right) after 24 hours of growth at 37° C. shaking.

FIG. 3A-3D. FIG. 3A depicts the percentage of L. monocytogenes strains that colocalized with the autophagy receptor p62 in infected bone marrow-derived macrophages (BMMs). FIG. 3B-3C depict intracellular growth curves of L. monocytogenes strains in BMMs. (B) shows growth curves of indicated L. monocytogenes strains in wild-type BMMs. (C) shows growth curves of indicated flavin-starved L. monocytogenes strains in riboflavin-deprived wild-type BMMs (for 3 hours) supplemented with 1 μM riboflavin just prior to infection. FIG. 3D depicts the generation time of intracellularly growing L. monocytogenes strains in riboflavin-sufficient and deprived BMMs between 2 to 5 hours.

FIG. 4A-4F. FIG. 4A-4C depict intracellular growth curves of L. monocytogenes strains in BMMs. (A) shows growth curves of indicated L. monocytogenes strains in wild-type BMMs. (B) shows growth curves of indicated L. monocytogenes strains in wild-type BMMs incubated with cell culture media containing excess (10 μM) riboflavin during infection. (C) shows growth curves of indicated flavin-starved L. monocytogenes strains in riboflavin-deficient wild-type BMMs. FIG. 4D-E depict cell death of wild-type (D) or AIM2 KO (E) BMMs infected with specified L. monocytogenes strains. FIG. 4F depicts intracellular growth curves of indicated L. monocytogenes strains in AIM2 KO BMMs.

FIG. 5A-5F. FIG. 5A-5C depict the broth growth curves of L. monocytogenes strains grown in chemically defined synthetic media with different flavin sources. (A) shows growth curves of wild-type L. monocytogenes grown in media with riboflavin, FMN, or FAD as the only flavin source. (B) shows growth curves of ΔribU mutant L. monocytogenes strain grown in media with riboflavin, FMN, or FAD as the sole flavin source. (C) shows growth curves of indicated L. monocytogenes strains grown in media with riboflavin. FIG. 5D shows intracellular growth curves of L. monocytogenes strains in murine BMMs. FIG. 5E-5F shows the bacterial burdens in the spleens (C) and livers (D) of CD-1 mice infected intravenously with 1×10⁵ CFUs of indicated L. monocytogenes strains 48 hours after infection.

FIG. 6A-6C. FIG. 6A depicts in vitro growth of L. monocytogenes strains in defibrinated sheep's blood. FIG. 6B depicts the bacterial burdens in the gallbladder of CD-1 mice infected intravenously with 1×10⁵ CFUs of indicated L. monocytogenes strains 48 hours after infection. FIG. 6C depicts the bacterial burdens in the gastrointestinal tract of CD-1 mice infected orally with 1×10⁸ CFUs of indicated L. monocytogenes strains.

FIG. 7 is a diagram of the riboflavin operon introduced into the L. monocytogenes chromosome with both pHyper (constitutive promoter) and pNative (inducible, native promoter from B. subtilis) depicted.

FIG. 8 depicts intracellular growth curves of L. monocytogenes strains in murine BMMs.

FIG. 9 depicts the bacterial burdens in the spleens and livers of B6 mice infected intravenously with 1×10³ CFUs of indicated L. monocytogenes strains 96 hours after infection.

FIG. 10A-10D depict the effect of infection with attenuated riboflavin-producing L. monocytogenes strains in ActA-minus backgrounds.

FIG. 11A-11B depict the effect of perforin-minus MAIT cells in riboflavin-producing L. monocytogenes.

FIG. 12 depicts intracellular growth curves of L. monocytogenes strains in wild-type murine bone marrow-derived macrophages (BMMs).

FIG. 13 depicts the bacterial burdens in the spleens and livers of CD-1 mice infected intravenously with various L. monocytogenes strains 47 hours after infection.

FIG. 14 depicts growth ΔactAΔinlB, ΔactAΔinlB+ΔribCΔribF, and ΔactAΔinlB+ΔribCΔribF+eetB::tn strains of L. monocytogenes in defibrinated sheep's blood.

FIG. 15 depicts the effect of vaccination with ΔactA and ΔactAΔribCΔribF mutant strain of L. monocytogenes.

### DETAILED DESCRIPTION

The present disclosure provides variant Listeria bacteria that lack functional ribC and ribF genes. Also provided are methods of making and using the variant Listeria bacteria, e.g., as vectors, vaccines, and therapeutics. The present disclosure provides variant Listeria bacteria that provide for production of a Mucosal-Associated Invariant T (MAIT) cell ligand in vivo. Also provided are methods of making and using the variant Listeria bacteria, e.g., to stimulate and expand the number of MAIT cells in an individual.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a variant Listeria bacterium” includes a plurality of such variant Listeria bacteria and reference to “the immunogenic composition” includes reference to one or more immunogenic compositions and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Variant Listeria Bacteria Comprising a Mutation(s) in a Gene(s) Required for FMN and FAD Biosynthesis

The present disclosure provides variant Listeria bacteria comprising a mutation in genes required for flavin mononucleotide (FMN) and flavin adenine dinucleotide (FAD) biosynthesis, where the variant Listeria is a conditionally obligate intracellular bacterium in vivo (i.e., is not capable of growing extracellularly in a mammal). While the variant Listeria can be grown (cultured) in vitro in a culture medium supplemented with FMN and FAD, it cannot grow in vivo (e.g., inside a mammal) extracellularly. A variant Listeria of the present disclosure requires flavin mononucleotide and flavin adenine dinucleotide supplementation to grow, and thus is a conditionally obligate intracellular bacterium when in vivo (e.g., when in a mammal or other animal host).

Genes required for FMN and FAD biosynthesis include ribC and ribF. The present disclosure provides variant Listeria bacteria that lack functional ribC and ribF genes. A variant Listeria bacterium can be generated by genetically modifying a parental Listeria cell such that the ribC and ribF genes are non-functional. A non-functional gene can be one that is completely deleted; a gene that is from 5% to 95% deleted (i.e., from 5% to 95% of the nucleotides are deleted, compared to a corresponding wild-type gene); a gene that has an insertion of one or more nucleotides (e.g., from 1 to 10, from 10 to 100, or more than 100, nucleotides), relative to a corresponding wild-type gene, where the insertion of one or more nucleotides renders the gene non-functional; and the like. In some cases, from 5% to 100% (e.g., from 5% to 10%, from 10% to 20%, from 20% to 30%, from 30% to 40%, from 40% to 50%, from 50% to 60%, from 60% to 70%, from 70% to 80%, from 80% to 90%, or from 90% to 100%) of a ribC gene and/or a ribF gene is deleted to generate a variant Listeria of the present disclosure.

In some cases, a variant Listeria bacterium of the present disclosure is a ΔribC/ΔribF bacterium, e.g., the ribC and ribF genes are deleted. RibC is a bifunctional enzyme that catalyzes the phosphorylation of riboflavin to FMN and the adenylylation of FMN to form FAD. RibF also converts FMN to FAD by adenylylation.

The Listeria cell (e.g., parental Listeria cell) that is used to generate a variant Listeria bacterium of the present disclosure can be any one of a number of different Listeria spp, and is typically a riboflavin auxotroph. Listeria spp of interest include, but are not limited to: L. fleischmannii, L. innocua, L. ivanovii, L. marthii, L. monocytogenes, L. rocourtiae, L. seeligeri, L. weihenstephanensis, and L. welshimeri. Thus, strains of Listeria other than L. monocytogenes may be host cells. In certain cases, the Listeria strain is L. monocytogenes.

In some instances, the Listeria host cell is attenuated. “Attenuation” and “attenuated” encompasses a Listeria host cell that is modified to reduce virulence. The host can be a human or animal host, or an organ, tissue, or cell. The Listeria host cell, to give a non-limiting example, can be attenuated to reduce binding to a host cell (e.g., human or non-human animal cell, such as a human or non-human mammalian cell), to reduce spread from one host cell to another host cell, to reduce extracellular growth, or to reduce intracellular growth in a host cell. Attenuation can be assessed by measuring, e.g., an indicum or indicia of virulence, the LD50, the rate of clearance from an organ, or the competitive index (see, e.g., Auerbuch, et al. (2001) Infect. Immunity 69:5953-5957). Generally, an attenuation results in an increase in the LD50 (the lethal dose, 50%; the dose (number of bacteria) required to kill half the members of a tested population after a specified test duration) and/or an increase in the rate of clearance by at least 25%; more generally by at least 50%; most generally by at least 100% (2-fold); normally by at least 5-fold; more normally by at least 10-fold; most normally by at least 50-fold; often by at least 100-fold; more often by at least 500-fold; and most often by at least 1000-fold; usually by at least 5000-fold; more usually by at least 10,000-fold; and most usually by at least 50,000-fold; and most often by at least 100,000-fold. Attenuation can also be assessed by determining the number of colony-forming units (CFUs).

In certain embodiments, attenuated Listeria according to the present disclosure are ones that exhibit a decreased virulence compared to a corresponding wild type strain in the Competitive Index Assay as described in Auerbach et al., “Development of a Competitive Index Assay To Evaluate the Virulence of Listeria monocytogenes actA Mutants during Primary and Secondary Infection of Mice,” Infection and Immunity, September 2001, p. 5953-5957, Vol. 69, No. 9. In this assay, mice are inoculated with test and reference, e.g., wild-type, strains of bacteria. Following a period of time, e.g., 48 to 60 hours, the inoculated mice are sacrificed and one or more organs, e.g., liver, spleen, are evaluated for bacterial abundance. In these embodiments, a given bacterial strain is considered to be less virulent if its abundance in the spleen is at least about 50-fold, or more, such as 70-fold or more less than that observed with the corresponding wild-type strain, and/or its abundance in the liver is at least about 10-fold less, or more, such as 20-fold or more less than that observed with the corresponding wild-type strain.

In yet other embodiments, bacteria are considered to be less virulent if they show abortive replication in less than about 8 hours, such as less than about 6 hours, including less than about 4 hours, as determined using the assay described in Jones and Portnoy, Intracellular growth of bacteria. (1994b) Methods Enzymol. 236:463-467. In yet other embodiments, bacteria are considered to be attenuated or less virulent if, compared to wild-type, they form smaller plaques in the plaque assay employed in U.S. Pat. No. 7,794,728 (the disclosure of which is herein incorporated by reference) where cells, such as murine L2 cells, are grown to confluency, e.g., in six-well tissue culture dishes, and then infected with bacteria. Subsequently, DME-agar containing gentamicin is added and plaques are grown for a period of time, e.g., 3 days. Living cells are then visualized by adding an additional DME-agar overlay, e.g., containing neutral red (GIBCO BRL) and incubated overnight. In such an assay, the magnitude in reduction in plaque size observed with the attenuated mutant as compared to the wild-type is, in certain embodiments, 10%, including 15%, such as 25% or more.

Attenuated bacteria may include one or more different mutations which confer the attenuated phenotype, where mutations of interest include hly mutations and/or lplA mutations, e.g., as described in U.S. Pat. No. 7,794,728 (the disclosure of which is herein incorporated by reference); actA and/or internalin B (InlB) mutations, e.g., as reported in Dung et al., Clin. Cancer Res. (2012) 18:858-868); etc. Thus, in some cases, a variant Listeria of the present disclosure comprises, in addition to a mutation(s) that renders ribC and ribF genes non-functional (e.g., by deletion of all or a portion of the ribC and ribF genes), a mutation in actA (encoding acting assembly-inducing protein ActA) and/or inlB (encoding InternalinB). In some cases, a variant Listeria of the present disclosure comprises: a) a deletion of all or a portion of ribC, which deletion renders the ribC gene non-functional; b) a deletion of all or a portion of ribF, which deletion renders the ribF gene non-functional; c) a deletion of all or a portion of actA, which deletion renders the actA gene non-functional; and d) a deletion of all or a portion of inlB, which deletion renders the inlB gene non-functional.

A variant Listeria bacterium of the present disclosure may include, in addition to the ribC/ribF modification described above, a deletion of all or a part of the eetB gene. Deletion of all or a part of the eetB gene can provide a growth advantage. In some cases, such a variant strain exhibits increased growth rate in broth in vitro, compared to the growth rate in broth of a Listeria bacterium comprising the ribC/ribF modification without the deletion of the eetB gene. In some cases, a variant Listeria of the present disclosure comprises: a) a deletion of all or a portion of ribC, which deletion renders the ribC gene non-functional; b) a deletion of all or a portion of ribF, which deletion renders the ribF gene non-functional; and c) a deletion of all or a part of eetB, which deletion renders the eetB gene non-functional. In some cases, a variant Listeria of the present disclosure comprises: a) a deletion of all or a portion of ribC, which deletion renders the ribC gene non-functional; b) a deletion of all or a portion of ribF, which deletion renders the ribF gene non-functional; c) a deletion of all or a portion of actA, which deletion renders the actA gene non-functional; d) a deletion of all or a portion of inlB, which deletion renders the inlB gene non-functional; and e) a deletion of all or a part of eetB, which deletion renders the eetB gene non-functional.

A variant Listeria bacterium of the present disclosure may include one or more genetic modifications in addition to the ribC/ribF modification described above, which one or more additional modifications provide for desirable qualities in the host cell, e.g., attenuation, enhanced immunogenicity, etc. Examples of such additional modifications include, but are not limited to, those described in PCT Published Application Nos.: WO 2014/106123; WO 2014/074635; WO 2009/143085; WO 2008027560WO 2008066774; WO 2007117371; WO 2007103225; WO 2005071088; WO 2003102168; WO 2003/092600; WO/2000/009733; and WO 1999/025376; the disclosures of which applications are herein incorporated by reference.

A variant Listeria bacterium of the present disclosure may include, in addition to the ribC/ribF modification described above, an Lm-RIID (L. monocytogenes recombinase-induced intracellular death) mutation. See, e.g., U.S. Pat. No. 9,511,129.

The bacteria may be live or Killed But Metabolically Active (“KBMA”). KBMA vaccine strains are constructed by abrogating the capacity for nucleotide excision repair through deletion of DNA repair genes such as uvrA and uvrB. The gene deletion renders the bacteria sensitive to photochemical inactivation through the combined treatment of psoralens and UVA. Because of their inability to repair the psoralen-induced DNA cross-links formed, KBMA bacterial strains are unable to replicate and are thus functionally noninfectious. This characteristic provides an improved safety profile in comparison to live attenuated strains. The very limited number of cross-links, however, preserves their metabolic activity, including antigen expression, and thus their immune potential. KBMA vaccine strains are described in U.S. Pat. No. 7,833,775, the disclosure of which is herein incorporated by reference.

In certain instances, a variant Listeria bacterium of the present disclosure expresses a heterologous antigen. The heterologous antigen is, in certain embodiments, one that is capable of providing protection in an animal against challenge by the infectious agent from which the heterologous antigen was derived, or which is capable of affecting tumor growth and metastasis in a manner which is of benefit to a host organism. Heterologous antigens which may be introduced into a Listeria strain of the subject disclosure by way of DNA encoding the same thus include any antigen which when expressed by Listeria serves to elicit a cellular immune response which is of benefit to the host in which the response is induced. Heterologous antigens therefore include those specified by infectious agents, wherein an immune response directed against the antigen serves to prevent or treat disease caused by the agent. Such heterologous antigens include, but are not limited to, viral, bacterial, fungal or parasite surface proteins and any other proteins, glycoproteins, lipoprotein, glycolipids, and the like. Heterologous antigens include tumor antigens. Heterologous antigens also include those which provide benefit to a host organism which is at risk for acquiring or which is diagnosed as having a tumor that expresses the heterologous antigen(s). The host organism is maybe a mammal, such as a human.

By the term “heterologous antigen.” as used herein, is meant a protein or peptide, a glycoprotein or glycopeptide, a lipoprotein or lipopeptide, or any other macromolecule which is not normally expressed in Listeria, which substantially corresponds to the same antigen in an infectious agent, a tumor cell or a tumor-related protein. The heterologous antigen is expressed by a strain of Listeria according to the present disclosure, and is processed and presented to cytotoxic T-cells upon infection of mammalian cells by the strain. The heterologous antigen expressed by Listeria species need not precisely match the corresponding unmodified antigen or protein in the tumor cell or infectious agent so long as it results in a T-cell response that recognizes the unmodified antigen or protein which is naturally expressed in the mammal. In other examples, the tumor cell antigen may be a mutant form of that which is naturally expressed in the mammal, and the antigen expressed by the Listeria species will conform to that tumor cell mutated antigen. By the term “tumor-related antigen,” as used herein, is meant an antigen which affects tumor growth or metastasis in a host organism. The tumor-related antigen may be an antigen expressed by a tumor cell, or it may be an antigen which is expressed by a non-tumor cell, but which when so expressed, promotes the growth or metastasis of tumor cells. The types of tumor antigens and tumor-related antigens which may be introduced into Listeria by way of incorporating DNA encoding the same, include any known or heretofore unknown tumor antigen. In other examples, the “tumor-related antigen” has no effect on tumor growth or metastasis, but is used as a component of the Listeria vaccine because it is expressed specifically in the tissue (and tumor) from which the tumor is derived. In still other examples, the “tumor-related antigen” has no effect on tumor growth or metastasis, but is used as a component of the Listeria vaccine because it is selectively expressed in the tumor cell and not in any other normal tissues.

The heterologous antigen useful in vaccine development may be selected using knowledge available to the skilled artisan, and many antigenic proteins which are expressed by tumor cells or which affect tumor growth or metastasis or which are expressed by infectious agents are currently known. For example, viral antigens which may be considered as useful as heterologous antigens include but are not limited to the nucleoprotein (NP) of influenza virus and the gag protein of HIV. Other heterologous antigens include, but are not limited to, HIV env protein or its component parts gp120 and gp41, HIV nef protein, and the HIV pol proteins, reverse transcriptase and protease. Still other heterologous antigens can be those related to hepatitis C virus (HCV), including but not limited to the E1 and E2 glycoproteins, as well as non-structural (NS) proteins, for example NS3. In addition, other viral antigens such as herpesvirus proteins may be useful. The heterologous antigens need not be limited to being of viral origin. Parasitic antigens, such as, for example, malarial antigens, are included, as are fungal antigens, bacterial antigens and tumor antigens.

As noted herein, a number of proteins expressed by tumor cells are also known and are of interest as heterologous antigens which may be inserted into the vaccine strain of the invention. These include, but are not limited to, the ber/abl antigen in leukemia, HPVE6 and E7 antigens of the oncogenic virus associated with cervical cancer, the MAGE1 and MZ2-E antigens in or associated with melanoma, and the MVC-1 and HER-2 antigens in or associated with breast cancer. Suitable heterologous antigens include cancer-associated antigens such as, e.g., carcinoembryonic antigen (CEA); epithelial glycoprotein-2 (EGP-2); epithelial glycoprotein-40 (EGP-40); folate binding protein (FBP); fetal acetylcholine receptor; ganglioside antigen GD2; Her2/neu; IL-13R-a2; kappa light chain; LeY; L1 cell adhesion molecule; melanoma-associated antigen (MAGE); MAGE-A1; mesothelin; MUC1; NKG2D ligands; oncofetal antigen (h5T4); prostate stem cell antigen (PSCA); prostate-specific membrane antigen (PSMA); tumor-associate glycoprotein-72 (TAG-72); vascular endothelial growth factor receptor-2 (VEGF-R2);and epidermal growth factor receptor (EGFR) vIII polypeptide. Other coding sequences of interest include, but are not limited to, costimulatory molecules, immunoregulatory molecules, and the like.

Bacteria as described herein may be generated using a variety of different protocols. As such, generation of the subject attenuated bacteria may be accomplished in a number of ways that are well known to those of skill in the art, including deletion mutagenesis, insertion mutagenesis, point mutations, mutagenesis which results in the generation of frameshift mutations, mutations which effect premature termination of a protein, and mutation of regulatory sequences which affect gene expression. Mutagenesis can be accomplished using recombinant DNA techniques or using traditional mutagenesis technology using mutagenic chemicals or radiation and subsequent selection of mutants. Representative protocols of different ways to generate bacteria according to the present invention are provided in the Experimental Section, below.

The introduction of DNA encoding a heterologous antigen into a strain of Listeria may be accomplished, for example, by the creation of a recombinant Listeria in which DNA encoding the heterologous antigen is harbored on a vector, such as a plasmid for example, which plasmid is maintained and expressed in the Listeria species, and in whose antigen expression is under the control of prokaryotic promoter/regulatory sequences. Alternatively, DNA encoding the heterologous antigen may be stably integrated into the Listeria chromosome by employing, for example, transposon mutagenesis, homologous recombination, or integrase mediated site-specific integration (as described in application Ser. No. 10/136,860, the disclosure of which is herein incorporated by reference).

Several approaches may be employed to express the heterologous antigen in Listeria species as will be understood by one skilled in the art once armed with the present disclosure. In certain embodiments, genes encoding heterologous antigens are designed to either facilitate secretion of the heterologous antigen from the bacterium or to facilitate expression of the heterologous antigen on the Listeria cell surface.

In certain embodiments, a fusion protein which includes the desired heterologous antigen and a secreted or cell surface protein of Listeria is employed. Listerial proteins which are suitable components of such fusion proteins include, but are not limited to, listeriolysin O (LLO) and phosphatidylinositol-specific phospholipase (PI-PLC). A fusion protein may be generated by ligating the genes which encode each of the components of the desired fusion protein, such that both genes are in frame with each other. Thus, expression of the ligated genes results in a protein comprising both the heterologous antigen and the Listerial protein. Expression of the ligated genes may be placed under the transcriptional control of a Listerial promoter/regulatory sequence such that expression of the gene is effected during growth and replication of the organism. Signal sequences for cell surface expression and/or secretion of the fused protein may also be added to genes encoding heterologous antigens in order to effect cell surface expression and/or secretion of the fused protein. When the heterologous antigen is used alone (i.e., in the absence of fused Listeria sequences), it may be advantageous to fuse thereto signal sequences for cell surface expression and/or secretion of the heterologous antigen. The procedures for accomplishing this are well known in the art of bacteriology and molecular biology.

The DNA encoding the heterologous antigen which is expressed is, in some embodiments, preceded by a suitable promoter to facilitate such expression. The appropriate promoter/regulatory and signal sequences to be used will depend on the type of Listerial protein desired in the fusion protein and will be readily apparent to those skilled in the art of Listeria molecular biology. For example, L. monocytogenes promoter/regulatory and/or signal sequences which may be used to direct expression of a fusion protein include, but are not limited to, sequences derived from the Listeria hly gene which encodes LLO, the Listeria p60 (iap) gene, and the Listeria actA gene which encodes a surface protein necessary for L. monocytogenes actin assembly. Other promoter sequences of interest include the plcA gene which encodes PI-PLC, the Listeria mpl gene, which encodes a metalloprotease, and the Listeria inlA gene which encodes internalin, a Listeria membrane protein. The heterologous regulatory elements such as promoters derived from phage and promoters or signal sequences derived from other bacterial species may be employed for the expression of a heterologous antigen by the Listeria species. Another suitable promoter is the constitutive HyPer promoter; see, e.g., Reniere et al. (2016) PLOS Pathogens doi.org/10.1371/journal.ppat.1005741.

In certain embodiments, the attenuated Listeria includes a vector. The vector may include DNA encoding a heterologous antigen. In some instances, the vector is a plasmid that is capable of replication in Listeria. The vector may encode a heterologous antigen, wherein expression of the antigen is under the control of eukaryotic promoter/regulatory sequences, e.g., is present in an expression cassette. Typical plasmids having suitable promoters that are of interest include, but are not limited to, pCMVbeta comprising the immediate early promoter/enhancer region of human cytomegalovirus, and those which include the SV40 early promoter region or the mouse mammary tumor virus LTR promoter region.

As such, in certain embodiments, the subject bacteria include at least one coding sequence for heterologous polypeptide/protein, as described above. In some instances, the coding sequence is one that lacks introns, e.g., is a continuous open reading frame that has a sequence which is the same as a cDNA sequence which may be produced from chromosomal sequence. In some embodiments, this coding sequence is part of an expression cassette, which provides for expression of the coding sequence in the Listeria cell for which the vector is designed. The term “expression cassette” as used herein refers to an expression module or expression construct made up of a recombinant DNA molecule containing at least one desired coding sequence and appropriate nucleic acid sequences necessary for the expression of the operably linked coding sequence in a particular host organism, i.e., the Listeria cell for which the vector is designed, such as the promoter/regulatory/signal sequences identified above, where the expression cassette may include coding sequences for two or more different polypeptides, or multiple copies of the same coding sequence, as desired. As such, the size of the encoded product may vary greatly, and a broad spectrum of different products may be encoded by the expression cassettes present in the vectors of this embodiment.

As indicated above, the vector may include at least one coding sequence, where in certain embodiments the vectors include two or more coding sequences, where the coding sequences may encode products that act concurrently to provide a desired result. In general, the coding sequence may encode any of a number of different products and may be of a variety of different sizes, where the above discussion merely provides representative coding sequences of interest.

### Composition Comprising a Variant Listeria

The present disclosure provides a composition comprising a variant Listeria of the present disclosure (variant Listeria comprising one or more mutations in one or more genes required for FMN and FAD biosynthesis; e.g., a ΔribCΔribF variant). A composition of the present disclosure can comprise, in addition to a variant Listeria of the present disclosure, one or more of: a salt (e.g., NaCl, MgCl₂, KCl, MgSO₄, etc.), a buffering agent, and the like. In some instance, a composition of the present disclosure comprises, in addition to a variant Listeria of the present disclosure, saline. In some cases, a composition of the present disclosure further comprises a multispecific antibody (e.g., a bispecific antibody). A multispecific antibody is in some instances a bispecific T cell engaging (BiTE) antibody. A multispecific antibody can include a first antigen-binding site specific for a cancer-associated antigen and a second antigen-binding site specific for a T cell (e.g., a mucosal-associated invariant T (MAIT) cell, a γ/δ T cell, a CD8⁺ cytotoxic T cell, a natural killer (NK) cell).

The present disclosure provides an immunogenic composition (also referred to herein as a “a vaccine composition”) comprising a variant Listeria of the present disclosure. An immunogenic composition of the present disclosure can comprise: a) a variant Listeria of the present disclosure; and b) an antigen. Suitable antigens include, but are not limited to, cancer-associated antigens, pathogen-associated antigens (e.g. viral antigens, pathogenic protozoan antigens, and the like).

In some cases, a composition comprising a variant Listeria of the present disclosure (a variant Listeria comprising one or more mutations in one or more genes required for FMN and FAD biosynthesis; e.g., a ΔribCΔribF variant) comprises a unit dose of the variant Listeria. A unit dose of the variant Listeria can be in a range of from 10⁴ to 10¹⁰ bacteria per dose. For example, in some cases, an “effective amount” of a variant Listeria of the present disclosure (a variant Listeria comprising one or more mutations in one or more genes required for FMN and FAD biosynthesis; e.g., a ΔribCΔribF variant) is in a range of from 10⁴ to 5×10⁴, from 5×10⁴ to 10⁵, from 10⁵ to 5×10⁵, from 5×10⁵ to 10⁶, from 10⁶ to 5×10⁶, from 5×10⁶ to 10⁷, from 10⁷ to 5×10⁷, from 5×10⁷ to 10⁸, from 10⁸ to 10⁹, or from 10⁹ to 10¹⁰, bacteria per unit dose. The present disclosure further provides a kit comprising a unit dose of a variant Listeria.

### Utility

The above-described bacteria (variant Listeria comprising one or more mutations in one or more genes required for FMN and FAD biosynthesis; e.g., a ΔribCΔribF variant) find use in a number of different applications. Representative uses of the subject bacteria include, but are not limited to: (a) immunogens for generating antibodies to Listeria spp.; (b) adjuvant compositions in immunizing protocols; (c) vectors for introducing macromolecules, e.g., nucleic acids or proteins, into the cytoplasm of target cells; and (d) vaccine compositions, e.g., for eliciting or boosting a cellular immune response in a host. Each of these representative applications is now further described separately below. Uses for attenuated Listeria spp. are also described in U.S. Pat. Nos. 8,679,476; 8,277,797; 8,192,991; 7,842,289; 7,794,728; 7,749,510; 7,488,487; 7,425,449; 6,599,502; 6,504,020; 6,287,556; 6,099,848; 6,004,815; 5,830,702; and 5,643,599; the disclosures of which applications are herein incorporated by reference.

### Vaccines

The subject bacteria (variant Listeria comprising one or more mutations in one or more genes required for FMN and FAD biosynthesis; e.g., a ΔribCΔribF variant) find use as vaccines (also referred to herein as an “immunogenic composition”). The vaccines of the present disclosure are administered to a vertebrate by contacting the vertebrate with a sub-lethal dose of the attenuated Listeria vaccine, where contact typically includes administering the vaccine to the host. In some embodiments, the bacteria are provided in a pharmaceutically acceptable formulation. Administration can be oral, parenteral, intranasal, intramuscular, intradermal, intraperitoneal, intravascular, subcutaneous, direct vaccination of lymph nodes, administration by catheter or any one or more of a variety of well-known administration routes. In farm animals, for example, the vaccine may be administered orally by incorporation of the vaccine in feed or liquid (such as water). It may be supplied as a lyophilized powder, as a frozen formulation or as a component of a capsule, or any other convenient, pharmaceutically acceptable formulation that preserves the antigenicity of the vaccine. Any one of a number of well-known pharmaceutically acceptable diluents or excipients may be employed in the vaccines of the invention. Suitable diluents include, for example, sterile, distilled water, saline, phosphate buffered solution, and the like. The amount of the diluent may vary widely, as those skilled in the art will recognize. Suitable excipients are also well known to those skilled in the art and may be selected, for example, from A. Wade and P. J. Weller, eds., Handbook of Pharmaceutical Excipients (1994) The Pharmaceutical Press: London. The dosage administered may be dependent upon the age, health and weight of the patient, the type of patient, and the existence of concurrent treatment, if any. The vaccines can be employed in dosage forms such as capsules, liquid solutions, suspensions, or elixirs, for oral administration, or sterile liquid for formulations such as solutions or suspensions for parenteral, intranasal intramuscular, or intravascular use. In accordance with the invention, the vaccine may be employed, in combination with a pharmaceutically acceptable diluent, as a vaccine composition, useful in immunizing a patient against infection from a selected organism or virus or with respect to a tumor, etc. Immunizing a patient means providing the patient with at least some degree of therapeutic or prophylactic immunity against selected pathogens, cancerous cells, etc.

The subject vaccines find use in methods for eliciting or boosting a cellular immune response, e.g., a helper T cell or a cytotoxic T-cell response to a selected agent, e.g., pathogenic organism, tumor, etc., in a vertebrate, where such methods include administering an effective amount of the Listeria vaccine. The subject vaccines find use in methods for eliciting in a vertebrate an innate immune response that augments the antigen-specific immune response. Furthermore, the vaccines of the present invention may be used for treatment post-exposure or post diagnosis. In general, the use of vaccines for post-exposure treatment would be recognized by one skilled in the art, for example, in the treatment of rabies and tetanus. The same vaccine of the present invention may be used, for example, both for immunization and to boost immunity after exposure. Alternatively, a different vaccine of the present invention may be used for post-exposure treatment, for example, such as one that is specific for antigens expressed in later stages of exposure. As such, the subject vaccines prepared with the subject vectors find use as both prophylactic and therapeutic vaccines to induce immune responses that are specific for antigens that are relevant to various disease conditions.

The patient may be any human and non-human animal susceptible to infection with the selected organism. The subject vaccines will find particular use with vertebrates such as mammals (including humans and non-human mammals); and with domestic animals. Domestic animals include domestic fowl, bovine, porcine, ovine, equine, caprine, canine, feline, Leporidate (such as rabbits), or other non-human animal.

The subject vaccines find use in vaccination applications as described in PCT Published Application Nos.: WO 2014/106123; WO 2014/074635; WO 2009/143085; WO 2008027560 WO 2008066774; WO 2007117371; WO 2007103225; WO 2005071088; WO 2003102168; WO 2003/092600; WO/2000/009733; and WO 1999/025376; the disclosures of which applications are herein incorporated by reference.

### Adjuvant Composition

The subject bacterial strains (variant Listeria comprising one or more mutations in one or more genes required for FMN and FAD biosynthesis; e.g., a ΔribCΔribF variant) also find use as immunopotentiating agents, i.e., as adjuvants. In such applications, the subject attenuated bacteria may be administered in conjunction with an immunogen, e.g., a tumor antigen, modified tumor cell, etc., according to methods known in the art where live bacterial strains are employed as adjuvants. See, e.g., Berd et al., Vaccine 2001 Mar. 21;19(17-19):2565-70.

In some embodiments, the bacterial strains are employed as adjuvants by chemically coupled to a sensitizing antigen. The sensitizing antigen can be any antigen of interest, where representative antigens of interest include, but are not limited to: viral agents, e.g., Herpes simplex virus; malaria parasite; bacteria, e.g., staphylococcus aureus bacteria, diphtheria toxoid, tetanus toxoid, shistosomula; tumor cells, e.g. CAD₂ mammary adenocarcinoma tumor cells, and hormones such as thyroxine T₄, triiodothyronine T₃, and cortisol. The coupling of the sensitizing antigen to the immunopotentiating agent can be accomplished by means of various chemical agents having two reactive sites such as, for example, bisdiazobenzidine, glutaraldehyde, di-iodoacetate, and diisocyanates, e.g., m-xylenediisocyanate and toluene-2,4-diisocyanate. Use of Listeria spp. as adjuvants is further described in U.S. Pat. No. 4,816,253; the disclosure of which is herein incorporated by reference.

### Delivery Vehicles

The subject bacteria (variant Listeria comprising one or more mutations in one or more genes required for FMN and FAD biosynthesis; e.g., a ΔribCΔribF variant) also find use as vectors or delivery vehicles for delivery of macromolecules into target cells, e.g., as described in: PCT publication no. WO 00/09733 (the disclosure of which is herein incorporated by reference); and Dietrich et al., Nature Biotechnology (1998) 16:181-185. A variety of different types of macromolecules may be delivered, including, but not limited to: nucleic acids, polypeptides/proteins, etc., as described in these publications.

### Variant Listeria that Produces a MAIT Cell Ligand

The present disclosure provides a variant Listeria that provides for production of the mucosal-associated invariant T (MAIT) cell ligand 5-(2-oxopropylideneamino)-6-D-ribitylaminouracil (5-OP-RU) and that activate and stimulate MAIT cells. In some cases, a variant Listeria of the present disclosure that produces a MAIT cell ligand also produces riboflavin. The present disclosure provides a variant Listeria that produces riboflavin (i.e., synthesizes riboflavin de novo) and that activate and stimulate proliferation of MAIT cells in vivo. A riboflavin-producing, MAIT cell stimulating, variant Listeria of the present disclosure is also referred to herein as a “ribDEAHT variant Listeria.” A ribDEAHT variant Listeria of the present disclosure is genetically modified to include a riboflavin operon (“ribDEAHT operon”) from a bacterium other than Listeria. Thus, a ribDEAHT variant Listeria of the present disclosure is genetically modified to include a heterologous ribDEAHT operon (i.e., a ribDEAHT operon from a bacterium other than Listeria. A heterologous ribDEAHT operon can be from any riboflavin-synthesizing bacterium. For example, a heterologous ribDEAHT operon can be from a Gram-positive bacterium such as Bacillus spp, such as B. subtilis. As depicted in FIG. 7, a heterologous ribDEAHT can include ribD, ribE, ribA, ribH, and ribT genes; these genes can be operably linked to a transcriptional control element (e.g., a promoter), where the promoter can be constitutive or can be inducible.

In some cases, a variant Listeria of the present disclosure that provides for production of the MAIT cell ligand 5-OP-RU (and that can activate and stimulate proliferation of MAIT cells) does not produce riboflavin. An example of such a variant Listeria is one that is genetically modified with a heterologous ribD gene and a heterologous ribA gene. Such a variant Listeria is referred to herein as a “ribDA variant Listeria.”

A ribDEAHT variant Listeria and a ribDA variant Listeria of the present disclosure are collectively referred to herein as “5-OP-RU precursor producing variant Listeria.” A 5-OP-RU precursor producing variant Listeria of the present disclosure produces the precursor 5-amino-6-(D-ribitylamino)uracil (5-A-RU), which reacts with an endogenous electrophile called methylglyoxal or glyoxal to form 5-OP-RU.

In some cases, a 5-OP-RU precursor producing variant Listeria of the present disclosure also has a mutation in a gloA gene (e.g., is a gloA strain). See, e.g., Anaya-Sanchez et al. (2021) PLOS Pathogens 17:e1009819.

In some cases, a ribDEAHT variant Listeria of the present disclosure has a deletion in a ribU gene such that it does not produce a RibU protein. Thus, e.g., in some cases, a ribDEAHT variant Listeria of the present disclosure is a AribU variant.

The Listeria host cell that is used to generate a ribDEAHT variant Listeria of the present disclosure or a ribDA variant Listeria of the present disclosure (i.e., a 5-OP-RU precursor producing variant Listeria of the present disclosure) can be any one of a number of different Listeria spp, and is typically a riboflavin auxotroph. Listeria spp of interest include, but are not limited to: L. fleischmannii, L. innocua, L. ivanovii, L. marthii, L. monocytogenes, L. rocourtiae, L. seeligeri, L. weihenstephanensis, and L. welshimeri. Thus, strains of Listeria other than L. monocytogenes may be host cells. In certain cases, the Listeria strain is L. monocytogenes.

In some instances, the Listeria host cell is attenuated. “Attenuation” and “attenuated” encompasses a Listeria host cell that is modified to reduce virulence. The host can be a human or animal host, or an organ, tissue, or cell. The Listeria host cell, to give a non-limiting example, can be attenuated to reduce binding to a host cell, to reduce spread from one host cell to another host cell, to reduce extracellular growth, or to reduce intracellular growth in a host cell. Attenuation can be assessed by measuring, e.g., an indicum or indicia of virulence, the LD50, the rate of clearance from an organ, or the competitive index (see, e.g., Auerbuch, et al. (2001) Infect. Immunity 69:5953-5957). Generally, an attenuation results an increase in the LD50 (the lethal dose, 50%; the dose (number of bacteria) required to kill half the members of a tested population after a specified test duration) and/or an increase in the rate of clearance by at least 25%; more generally by at least 50%; most generally by at least 100% (2-fold); normally by at least 5-fold; more normally by at least 10-fold; most normally by at least 50-fold; often by at least 100-fold; more often by at least 500-fold; and most often by at least 1000-fold; usually by at least 5000-fold; more usually by at least 10,000-fold; and most usually by at least 50,000-fold; and most often by at least 100,000-fold.

In certain embodiments, attenuated Listeria according to the present disclosure are ones that exhibit a decreased virulence compared to a corresponding wild type strain in the Competitive Index Assay as described in Auerbach et al., “Development of a Competitive Index Assay To Evaluate the Virulence of Listeria monocytogenes actA Mutants during Primary and Secondary Infection of Mice,” Infection and Immunity, September 2001, p. 5953-5957, Vol. 69, No. 9. In this assay, mice are inoculated with test and reference, e.g., wild-type, strains of bacteria. Following a period of time, e.g., 48 to 60 hours, the inoculated mice are sacrificed and one or more organs, e.g., liver, spleen, are evaluated for bacterial abundance. In these embodiments, a given bacterial strain is considered to be less virulent if its abundance in the spleen is at least about 50-fold, or more, such as 70-fold or more less than that observed with the corresponding wild-type strain, and/or its abundance in the liver is at least about 10-fold less, or more, such as 20-fold or more less than that observed with the corresponding wild-type strain.

In yet other embodiments, bacteria are considered to be less virulent if they show abortive replication in less than about 8 hours, such as less than about 6 hours, including less than about 4 hours, as determined using the assay described in Jones and Portnoy, Intracellular growth of bacteria. (1994b) Methods Enzymol. 236:463-467. In yet other embodiments, bacteria are considered to be attenuated or less virulent if, compared to wild-type, they form smaller plaques in the plaque assay employed in U.S. Pat. No. 7,794,728 (the disclosure of which is herein incorporated by reference) where cells, such as murine L2 cells, are grown to confluency, e.g., in six-well tissue culture dishes, and then infected with bacteria. Subsequently, DME-agar containing gentamicin is added and plaques are grown for a period of time, e.g., 3 days. Living cells are then visualized by adding an additional DME-agar overlay, e.g., containing neutral red (GIBCO BRL) and incubated overnight. In such an assay, the magnitude in reduction in plaque size observed with the attenuated mutant as compared to the wild-type is, in certain embodiments, 10%, including 15%, such as 25% or more.

Attenuated bacteria may include one or more different mutations which confer the attenuated phenotype, where mutations of interest include hly mutations and/or IplA mutations, e.g., as described in U.S. Pat. No. 7,794,728 (the disclosure of which is herein incorporated by reference); actA and/or internalin B (InlB) mutations, e.g., as reported in Dung et al., Clin. Cancer Res. (2012) 18:858-868); etc. Thus, in some cases, a ribDEAHT variant Listeria of the present disclosure comprises, in addition to a heterologous ribDEAHT operon, a mutation in actA and/or InlB.

A variant Listeria bacterium of the present disclosure may include one or more genetic modifications in addition to a heterologous ribDEAHT operon or heterologous ribDA genes, as described above, which one or more additional modifications provide for desirable qualities in the host cell, e.g., attenuation, enhanced immunogenicity, etc. Examples of such additional modifications include, but are not limited to, those described in PCT Published Application Nos.: WO 2014/106123; WO 2014/074635;WO 2009/143085; WO 2008027560 WO 2008066774; WO 2007117371; WO 2007103225; WO 2005071088; WO 2003102168; WO 2003/092600; WO/2000/009733; and WO 1999/025376; the disclosures of which applications are herein incorporated by reference.

The bacteria may be live or Killed But Metabolically Active (“KBMA”). KBMA vaccine strains are constructed by abrogating the capacity for nucleotide excision repair through deletion of DNA repair genes such as uvrA and uvrB. The gene deletion renders the bacteria sensitive to photochemical inactivation through the combined treatment of psoralens and UVA. Because of their inability to repair the psoralen-induced DNA cross-links formed, KBMA bacterial strains are unable to replicate and are thus functionally noninfectious. This characteristic provides an improved safety profile in comparison to live attenuated strains. The very limited number of cross-links, however, preserves their metabolic activity, including antigen expression, and thus their immune potential. KBMA vaccine strains are described in U.S. Pat. No. 7,833,775, the disclosure of which is herein incorporated by reference.

In certain instances, a ribDEAHT variant Listeria of the present disclosure or a ribDA variant Listeria of the present disclosure (a 5-OP-RU precursor-producing variant Listeria of the present disclosure) expresses a heterologous antigen. The heterologous antigen is, in certain embodiments, one that is capable of providing protection in an animal against challenge by the infectious agent from which the heterologous antigen was derived, or which is capable of affecting tumor growth and metastasis in a manner which is of benefit to a host organism. Heterologous antigens which may be introduced into a 5-OP-RU precursor producing variant Listeria of the present disclosure by way of DNA encoding the same thus include any antigen which when expressed by Listeria serves to elicit a cellular immune response which is of benefit to the host in which the response is induced. Heterologous antigens therefore include those specified by infectious agents, wherein an immune response directed against the antigen serves to prevent or treat disease caused by the agent. Such heterologous antigens include, but are not limited to, viral, bacterial, fungal or parasite surface proteins and any other proteins, glycoproteins, lipoprotein, glycolipids, and the like. Heterologous antigens also include those which provide benefit to a host organism which is at risk for acquiring or which is diagnosed as having a tumor that expresses the heterologous antigen(s). The host organism is maybe a mammal, such as a human.

By the term “heterologous antigen,” as used herein, is meant a protein or peptide, a glycoprotein or glycopeptide, a lipoprotein or lipopeptide, or any other macromolecule which is not normally expressed in Listeria, which substantially corresponds to the same antigen in an infectious agent, a tumor cell or a tumor-related protein. The heterologous antigen is expressed by a 5-OP-RU precursor producing variant Listeria of the present disclosure, and is processed and presented to cytotoxic T-cells upon infection of mammalian cells by the strain. The heterologous antigen expressed by a 5-OP-RU precursor producing variant Listeria of the present disclosure need not precisely match the corresponding unmodified antigen or protein in the tumor cell or infectious agent so long as it results in a T-cell response that recognizes the unmodified antigen or protein which is naturally expressed in the mammal. In other examples, the tumor cell antigen may be a mutant form of that which is naturally expressed in the mammal, and the antigen expressed by the Listeria species will conform to that tumor cell mutated antigen. By the term “tumor-related antigen,” as used herein, is meant an antigen which affects tumor growth or metastasis in a host organism. The tumor-related antigen may be an antigen expressed by a tumor cell, or it may be an antigen which is expressed by a non-tumor cell, but which when so expressed, promotes the growth or metastasis of tumor cells. The types of tumor antigens and tumor-related antigens which may be introduced into Listeria by way of incorporating DNA encoding the same, include any known or heretofore unknown tumor antigen. In other examples, the “tumor-related antigen” has no effect on tumor growth or metastasis, but is used as a component of the Listeria vaccine because it is expressed specifically in the tissue (and tumor) from which the tumor is derived. In still other examples, the “tumor-related antigen” has no effect on tumor growth or metastasis, but is used as a component of the Listeria vaccine because it is selectively expressed in the tumor cell and not in any other normal tissues.

The heterologous antigen useful in vaccine development may be selected using knowledge available to the skilled artisan, and many antigenic proteins which are expressed by tumor cells or which affect tumor growth or metastasis or which are expressed by infectious agents are currently known. For example, viral antigens which may be considered as useful as heterologous antigens include but are not limited to the nucleoprotein (NP) of influenza virus and the gag protein of HIV. Other heterologous antigens include, but are not limited to, HIV env protein or its component parts gp120 and gp41, HIV nef protein, and the HIV pol proteins, reverse transcriptase and protease. Still other heterologous antigens can be those related to hepatitis C virus (HCV), including but not limited to the E1 and E2 glycoproteins, as well as non-structural (NS) proteins, for example NS3. In addition, other viral antigens such as herpesvirus proteins may be useful. The heterologous antigens need not be limited to being of viral origin. Parasitic antigens, such as, for example, malarial antigens, are included, as are fungal antigens, bacterial antigens and tumor antigens.

As noted herein, a number of proteins expressed by tumor cells are also known and are of interest as heterologous antigens which may be inserted into the vaccine strain of the invention. These include, but are not limited to, the ber/abl antigen in leukemia, HPVE6 and E7 antigens of the oncogenic virus associated with cervical cancer, the MAGE1 and MZ2-E antigens in or associated with melanoma, and the MVC-1 and HER-2 antigens in or associated with breast cancer. Suitable heterologous antigens include cancer-associated antigens such as, e.g., carcinoembryonic antigen (CEA); epithelial glycoprotein-2 (EGP-2); epithelial glycoprotein-40 (EGP-40); folate binding protein (FBP); fetal acetylcholine receptor; ganglioside antigen GD2; Her2/neu; IL-13R-a2; kappa light chain; LeY; L1 cell adhesion molecule; melanoma-associated antigen (MAGE); MAGE-A1; mesothelin; MUC1; NKG2D ligands; oncofetal antigen (h5T4); prostate stem cell antigen (PSCA); prostate-specific membrane antigen (PSMA); tumor-associate glycoprotein-72 (TAG-72); vascular endothelial growth factor receptor-2 (VEGF-R2);and epidermal growth factor receptor (EGFR) vIII polypeptide. Other coding sequences of interest include, but are not limited to, costimulatory molecules, immunoregulatory molecules, and the like.

In some cases, a 5-OP-RU precursor producing variant Listeria of the present disclosure is genetically modified to produce a chimeric antigen receptor (CAR). A CAR can comprise an antigen-binding portion that binds to a cancer-associated antigen. The antigen-binding portion can be, e.g., a single-chain Fv, a nanobody, and the like.

### Composition Comprising a ribDEAHT Variant Listeria or a ribDA Variant Listeria

The present disclosure provides a composition comprising a variant Listeria of the present disclosure (a ribDEAHT variant Listeria of the present disclosure; a ribDA variant Listeria of the present disclosure). A composition of the present disclosure can comprise, in addition to a variant Listeria of the present disclosure, one or more of: a salt (e.g., NaCl, MgCl₂, KCl, MgSO₄, etc.), a buffering agent, and the like. In some instance, a composition of the present disclosure comprises, in addition to a variant Listeria of the present disclosure, saline. In some cases, a composition of the present disclosure further comprises a multispecific antibody (e.g., a bispecific antibody). A multispecific antibody is in some instances a bispecific T cell engaging (BiTE) antibody. A multispecific antibody can include a first antigen-binding site specific for a cancer-associated antigen and a second antigen-binding site specific for a T cell (e.g., a mucosal-associated invariant T (MAIT) cell, a γ/δ T cell, a CD8⁺ cytotoxic T cell).

The present disclosure provides an immunogenic composition (also referred to herein as a “a vaccine composition”) comprising a variant Listeria of the present disclosure. An immunogenic composition of the present disclosure can comprise: a) a variant Listeria of the present disclosure; and b) an antigen. Suitable antigens include, but are not limited to, cancer-associated antigens, pathogen-associated antigens (e.g. viral antigens, pathogenic protozoan antigens, and the like).

### Utility

A 5-OP-RU precursor producing variant Listeria of the present disclosure finds use in activating and/or stimulating proliferation of MAIT cells. MAIT cells are innate-like T cells defined by their semi-invariant αβ T cell receptor (TCR) which recognizes small-molecule biosynthetic derivatives of riboflavin synthesis presented on the restriction molecule major histocompatibility complex (MHC)-related protein-1 (MR1). MAIT cell ligands include 5-(2-oxopropylideneamino)-6-D-ribitylaminouracil (5-OP-RU) and 5-(2-oxoethylideneamino)-6-D-ribitylaminouracil (5-OE-RU), which are produced by a wide variety of bacteria, mycobacteria and yeasts during riboflavin (vitamin B2) synthesis. MAIT cells have an intrinsic effector-memory phenotype, e.g., CD45RA ⁻CD45RO⁺ CD95^{Hi}CD62^{Lo}CD44^{Hi}, and have the ability to secrete various pro-inflammatory cytokines.

In some cases, a 5-OP-RU precursor producing variant Listeria of the present disclosure is administered to an individual, where said administration results in increase in the number of MAIT cells in the individual. In some cases, administration of an effective amount of a 5-OP-RU precursor producing variant Listeria of the present disclosure to an individual results in an increase of at least 5%, at least 10%, at least 15%, at least 20%, at least 25%, at least 50%, or more than 50% in the number of MAIT cells in the individual. In some cases, administration of an effective amount of a 5-OP-RU precursor producing variant Listeria of the present disclosure to an individual results in activation of MAIT cells in the individual.

In some cases, a 5-OP-RU precursor producing variant Listeria of the present disclosure is administered orally to an individual to modulate an immune response in the intestines of the individual.

An “effective amount” of a 5-OP-RU precursor producing variant Listeria of the present disclosure of the present disclosure is an amount that, when administered to an individual in need thereof, produces a beneficial effect in the individual (e.g., a clinically beneficial effect). In some cases, an effective amount of a 5-OP-RU precursor producing variant Listeria of the present disclosure is an amount that, when administered to an individual in need thereof, increases the number of MAIT cells in the individual. In some cases, an effective amount of a 5-OP-RU precursor producing variant Listeria of the present disclosure is an amount that, when administered to an individual having a tumor, reduces the tumor volume and/or reduces the number of cancer cells in the individual.

In some cases, an “effective amount” of a 5-OP-RU precursor producing variant Listeria of the present disclosure is in a range of from 10⁴ to 10¹⁰ bacteria per dose. For example, in some cases, an “effective amount” of a 5-OP-RU precursor producing variant Listeria of the present disclosure is in a range of from 10⁴ to 5×10⁴, from 5×10⁴ to 10⁵, from 10⁵ to 5×10⁵, from 5×10⁵ to 10⁶, from 10⁶ to 5×10⁶, from 5×10⁶ to 10⁷, from 10⁷ to 5×10⁷, from 5×10⁷ to 10⁸, from 10⁸ to 10⁹, or from 10⁹ to 10¹⁰, bacteria per unit dose.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A variant Listeria bacterium comprising a mutation in gene required for flavin mononucleotide (FMN) and flavin adenine dinucleotide (FAD) biosynthesis, wherein the variant Listeria bacterium does not grow extracellularly in a mammal.

Aspect 2. The variant Listeria bacterium of aspect 1, wherein the gene required for FMN and FAD synthesis is a ribC gene and/or a ribF gene.

Aspect 3. The variant Listeria bacterium of aspect 1 or aspect 2, wherein the variant Listeria bacterium is a conditionally obligate intracellular bacterium.

Aspect 4. The variant Listeria bacterium of any one of aspects 1-3, wherein the variant Listeria bacterium requires flavin mononucleotide and flavin adenine dinucleotide supplementation to grow.

Aspect 5. The variant Listeria bacterium of any one of aspects 2-4, wherein the mutation comprises a deletion of all or a portion of the ribC gene and/or the ribF gene.

Aspect 6. The variant Listeria bacterium of any one of aspect 5, wherein the variant is genetically modified to comprise a heterologous nucleic acid comprising a nucleotide sequence encoding at least one heterologous gene product.

Aspect 7. The variant Listeria bacterium of aspect 6, wherein the heterologous nucleic acid is integrated into the bacterial genome.

Aspect 8. The variant Listeria bacterium of aspect 6 or aspect 7, wherein the at least one heterologous gene product comprises an antigen.

Aspect 9. The variant Listeria bacterium of aspect 8, wherein the antigen is a cancer-associated antigen.

Aspect 10. The variant Listeria bacterium of any one of aspects 1-9, wherein said variant Listeria bacterium is a variant Listeria monocytogenes bacterium.

Aspect 11. The variant Listeria bacterium of any one of aspects 1-10, wherein the variant Listeria bacterium further comprises one or more additional mutations that confers an attenuated phenotype on the bacterium and/or one or more additional mutations that provide a growth advantage.

Aspect 12. The variant Listeria bacterium of aspect 11, wherein the one or more additional mutations that confers an attenuated phenotype comprises a mutation in a gene selected from actA and inlB, optionally wherein the one or more additional mutations that provide a growth advantage comprises a mutation in an eetB gene.

Aspect 13. A composition comprising:
a) a variant Listeria bacterium of any one of aspects 1-12; and
b) a multispecific antibody.

Aspect 14. The composition of aspect 13, wherein the multispecific antibody comprises: i) a first antigen-binding site specific for a cancer-associated antigen; and ii) a second antigen-binding site specific for a T cell.

Aspect 15. The composition of aspect 14, wherein the T cell is a mucosal-associated invariant T (MAIT) cell, a γ/δ T cell, a CD8⁺ T cell, or a natural killer (NK) cell.

Aspect 16. An immunogenic composition comprising the variant Listeria of any one of aspects 1-12.

Aspect 17. A method of inducing an immune response in an individual, the method comprising administering to the individual an effective amount of an immunogenic composition according to aspect 16.

Aspect 18. The method of aspect 17, wherein the variant Listeria bacterium is genetically modified to comprise a heterologous nucleic acid comprising a nucleotide sequence encoding a heterologous polypeptide, and wherein said immune response is induced to the heterologous polypeptide.

Aspect 19. The method of aspect 18, wherein the heterologous polypeptide is an antigen.

Aspect 20. The method of aspect 18, wherein the antigen is a cancer-associated antigen.

Aspect 21. The method of any one of aspects 17-20, wherein said immune response comprises a gamma-delta T cell response.

Aspect 22. A kit comprising a unit dose of the immunogenic composition of aspect 16.

Aspect 23. The kit of aspect 22, wherein the unit dose is an oral dose.

Aspect 24. The kit of aspect 22, wherein the unit dose is injectable.

Aspect 25. The kit of any one of aspects 22-24, further comprising a recombinant expression vector comprising a nucleotide sequence encoding a heterologous antigen.

Aspect 26. A 5-OP-RU precursor-producing variant Listeria that provides for production of the Mucosal-Associated Invariant T (MAIT) cell ligand 5-(2-oxopropylideneamino)-6-D-ribitylaminouracil (5-OP-RU).

Aspect 27. The 5-OP-RU precursor-producing variant Listeria of aspect 26, wherein the variant Listeria comprises a heterologous ribDEAHT operon, wherein the variant Listeria synthesizes riboflavin and stimulates proliferation of MAIT cells.

Aspect 28. The 5-OP-RU precursor-producing variant Listeria of aspect 26, wherein the variant Listeria comprises heterologous ribD and ribA genes.

Aspect 29. The 5-OP-RU precursor-producing variant Listeria of any one of aspects 26-28, wherein the variant is genetically modified to comprise a heterologous nucleic acid comprising a nucleotide sequence encoding at least one heterologous gene product.

Aspect 30. The 5-OP-RU precursor-producing variant Listeria bacterium of aspect 29, wherein the heterologous nucleic acid is integrated into the bacterial genome.

Aspect 31. The 5-OP-RU precursor-producing variant Listeria bacterium of aspect 29 or aspect 30, wherein the at least one heterologous gene product comprises an antigen.

Aspect 32. The variant Listeria bacterium of aspect 31, wherein the antigen is a cancer-associated antigen.

Aspect 33. The variant Listeria bacterium of aspect 29 or aspect 30, wherein the at least one heterologous gene product comprises a chimeric antigen receptor.

Aspect 34. The variant Listeria bacterium of any one of aspects 26-33, wherein the variant Listeria bacterium further comprises one or more additional mutations that confers an attenuated phenotype on the bacterium and/or one or more additional mutations that provide a growth advantage.

Aspect 35. The variant Listeria bacterium of aspect 34, wherein the one or more additional mutations that confers an attenuated phenotype comprises a mutation in a gene selected from actA and inlB, and wherein the one or more additional mutations that provide a growth advantage comprises a mutation in an eetB gene.

Aspect 36. A composition comprising the variant Listeria of any one of aspects 26-35.

Aspect 37. A method of increasing the number and activation state of mucosal-associated invariant T cell (MAIT) cells in an individual, the method comprising administering to the individual an effective amount of the variant Listeria of any one of aspects 26-35.

Aspect 38. A method of treating cancer in an individual, the method comprising administering to the individual an effective amount of the variant Listeria of any one of aspects 26-35.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); i.v., intravenous (ly); s.c., subcutaneous (ly); and the like.

### Example 1: Obligate Intracellular Pathogenic Strains of L. monocytogenes

### Materials and Methods

### Bacterial Culture and Strains

Strains of L. monocytogenes (Table 1) were derived from the wild-type 10403S strain and were cultured in filter-sterilized nutrient-rich Brain Heart Infusion (BHI) media (BD, Sparks, MD, USA) containing 200 μg/mL streptomycin (Sigma-Aldrich, St. Louis, MO, USA). Construction of the ΔribU (lmo1945), ΔribC (Imo1329), ΔribF (Imo0728), and ΔribCΔribF strains was done using allelic exchange with the temperature sensitive plasmid pKSV7. During the process of generating the ΔribC, ΔribF, and double ribC/ribF mutant strains, the bacteria were always cultured in BHI media with 2.5 μM FMN (Sigma-Aldrich, St. Louis, MO, USA) and 2.5 μM FAD (Sigma-Aldrich, St. Louis, MO, USA) to circumvent synthetic lethality. For all procedures in which the ΔribC, ΔribF, and ΔribCΔribF strains were used, the bacteria were always grown in BHI media with 2.5 μM FMN and 2.5 μM FAD.

Generation of the ΔribU strain expressing the ribDEAHT operon or complemented strain expressing ribU was done by amplifying the ribDEAHT operon with its native promoter from Bacillus subtilis and the ribU gene with its native promoter from wild-type L. monocytogenes, respectively, and cloning them into the pPL2 integrating vector. Similarly, complementation of the ΔribCΔribF mutant was done by amplifying the ribC gene with its native promoter from wild-type L. monocytogenes and cloning it into the pPL2 vector. They were integrated into the L. monocytogenes genome via conjugation. Broth growth curves were performed with L. monocytogenes strains from overnight cultures grown at 37° C. shaking (200 rpm). Nutrient-rich (BHI) and chemically defined synthetic media growth curves were started at an optical density (OD600) of 0.03. Growth curves were spectrophotometrically measured by optical density at a wavelength of 600 nm (OD600).

### Tissue Cultures and Growth Media

Bone marrow-derived macrophages (BMMs) were prepared by collecting bone marrow from 8-weeks-old female wild-type (Jackson laboratory) and AIM2 KO (University of Massachusetts Medical School) C57BL/6J mice. BMMs were cultured in high glucose Gibco Dulbecco's Modified Eagle's Medium (DMEM) (Thermo Fisher Scientific, Waltham, MA, USA) with 20% fetal bovine serum (FBS) (Avantor-Seradigm, Radnor, PA, USA), 10% M-CSF-producing 3T3 cell supernatant, 1% L-glutamine (Corning, Lowell, MA, USA), 1% sodium pyruvate (Corning, 315 Lowell, MA, USA), 14 mM 2-mercaptoethanol (Gibco Thermo Fisher Scientific, Waltham, MA, USA), or with a modified DMEM recipe described in the experimental procedure.

### Intracellular Growth Curves

Sixteen to eighteen hours prior to infection, 3×10⁶ BMMs were seeded in 60 mm non-TC treated dishes (MIDSCI, St. Louis, MO, USA) containing 14 12 mm glass coverslips (Thermo Fischer Scientific, Waltham, MA, USA) in each dish. L. monocytogenes strains were grown at 30° C. overnight in 14 ml round polypropylene tubes (Thermo Fisher Scientific, Waltham, MA, USA) at a slanted position. The bacteria were washed and diluted in sterile 1X phosphate-buffered saline (PBS) and BMMs were infected at a multiplicity of infection (MOI) of 0.25. Half an hour post-infection, the cells were washed twice with 1X PBS. One-hour post-infection, 50 μg/mL of gentamicin sulfate (Sigma Aldrich, St. Louis, MO, USA) was added to the cell culture media to kill/prevent bacteria from growing extracellularly.

### Intracellular Growth Curves in Media Lacking Riboflavin

To deplete the intracellular flavins in L. monocytogenes, bacterial cultures were started 2 days prior to BMM infection in chemically defined media containing 1 μM riboflavin and grown at 37° C. with shaking. The bacteria were washed twice 16-18 hours prior to infection, with 1X PBS and then diluted into chemically defined media lacking flavins and grown at 37° C. with shaking.

Macrophages were washed twice, 3 hours prior to BMM infection, with 1X PBS and the cell culture media was replaced with DMEM high glucose lacking riboflavin (Millipore Sigma, Burlington, MA, USA), with 20% dialyzed FBS-using SnakeSkin dialysis tubing, 3.5K MWCO (Thermo Fisher Scientific, Waltham, MA, USA), and other components as described in the tissue culture and growth media section above. The riboflavin-starved L. monocytogenes were washed and diluted in sterile 1X PBS and the BMMs were infected at an MOI of 0.25. These growth curves were performed without the addition of riboflavin, unless otherwise stated in the legend.

### Cell Death (Lactate Dehydrogenase Release) Assay

Sixteen to eighteen hours before infection, 5×10⁵ BMMs/well were seeded in 24-well plates with 100 ng/ml of Pam3CSK4 (InvivoGen, San Diego, CA, USA) in DMEM media. Before infecting the BMMs, the cell culture media was replaced with DMEM media with 5% FBS. L. monocytogenes strains were grown overnight, slanted, at 30° C. For the infection, bacteria were diluted in 1X PBS and BMMs were infected at an MOI of 4. Half an hour post-infection, the BMMS were washed twice with 1X PBS and DMEM media with 5% FBS and 50 μg/mL of gentamicin was added to wells.

### Mouse Intravenous Infections

Eight-week-old female CD-1 mice (Charles River Laboratories, Wilmington, MA, USA) were infected via the tail vain with 200 μL of PBS containing 1×10⁵ logarithmically growing bacteria. The mice were euthanized 48 hours post-infection and the spleen, liver, and gallbladder were collected, homogenized, and plated to determine the number of CFU per organ.

### Blood Growth Curve

The growth of L. monocytogenes strains in blood was determined using defibrinated sheep's blood (HemoStat Laboratories, Dixon, CA, USA). The bacteria were grown logarithmically for 2.5 hours, washed, and resuspended in 3 mL of defibrinated sheep's blood at a concentration of 1×10⁶/mL. Blood cultures were incubated at 37° C. shaking. The growth of L. monocytogenes in blood was monitored for 3 days by diluting the blood in 1X PBS and plating to determine the number of CFU in total blood.

### Mouse Oral Infections

Mice were given 5 mg/mL streptomycin sulfate salt (Sigma-Aldrich, St. Louis, MO, USA) in the drinking water, 48 hours prior to infection. Mice were transferred to clean cages 18-24 hours prior to infection and the food source (mouse colony chow) was removed to start the overnight fast. The day of the infection, a 3 mm piece of bread was inoculated with 1×10⁸ logarithmically growing bacteria in 1X PBS and covered with 3 μL of butter. Each 8-week-old female CD-1 mouse (Charles River Laboratories, Wilmington, MA, USA) was then fed a single piece of infected bread. The streptomycin sulfate water was replaced with standard drinking water and the chow was restored. Fecal samples were collected everyday post-infection for 5 days, weighted, vortexed at 4° C. for 10 min, and plated to determine the number of CFU per gram of stool.

### Phagosomal Escape Assay

BMMs were seeded in 24-well plates containing 12 mm glass coverslips (Thermo Fischer Scientific, Waltham, MA, USA) and cultured overnight. BMMs were treated with 250 ng/mL of cytochalasin D (Sigma-Aldrich, St. Louis, MO, USA) 30 min prior to infection (MOI of 15) with L. monocytogenes strains grown at 30° C. overnight in a slanted position. At 1 hour 15 min post infection, the BMMs were washed twice with 1X PBS and fixed with 4% paraformaldehyde (Electron Microscopy Sciences, Hatfield, PA, USA) for 15 min. The immunofluorescence staining, microscopy, and image analysis then proceeded. The primary antibodies used were rabbit anti-Listeria (BD Difco, Franklin Lakes, NJ, USA; cat. no. 223021) at a 1:1000 dilution, and guinea pig anti-p62 (Fitzgerald, Acton, MA, USA; cat. no. 20R-PP001) at a 1:200 dilution. The secondary antibodies used were Rhodamine Red-X goat anti-rabbit IgG (Invitrogen-Thermo Fisher Scientific, Waltham, MA, USA; cat. no. R6394) at 1:2000 dilution and AlexaFluor-647 goat anti-guinea pig IgG (Invitrogen-Thermo Fisher Scientific, Waltham, MA, USA; cat. no. A21450) at a 1:2000 dilution. At least 100 bacteria per condition were quantified for analysis.

### Results

### RibU is Required for Virulence in Mice

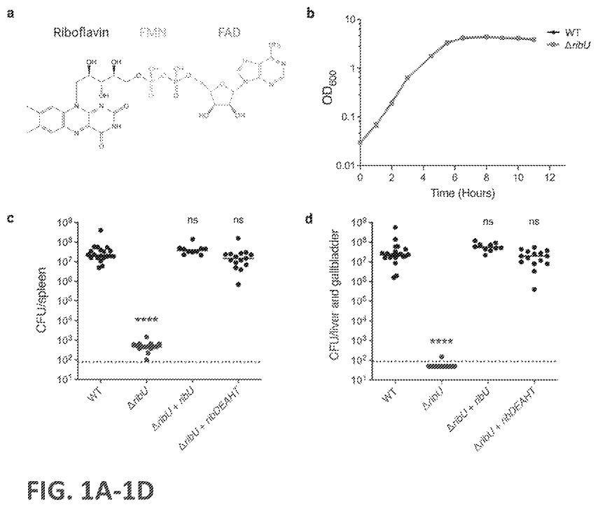

A mutant strain of L. monocytogenes with a transposon insertion in the ribU gene, the sole annotated riboflavin transporter in L. monocytogenes, was isolated. To confirm that RibU was not essential for growth, and assess flavin acquisition and requirements during pathogenesis, a L. monocytogenes strain with an in-frame deletion in ribU (ΔribU) was generated. Like the transposon mutant, the ΔribU strain had no detectable growth defect in nutrient-rich media compared to wild-type L. monocytogenes (FIG. 1b). In contrast, the AribU strain had a 5-log virulence defect in the spleens of mice compared to wild-type L. monocytogenes (FIG. 1c), and no colony-forming units (CFUs) could be recovered from the livers of infected mice (FIG. 1d). Complementation of the ΔribU mutant with a ribU gene with its endogenous promoter (ΔribU+ribU) fully restored virulence in vivo (FIG. 1c,d).

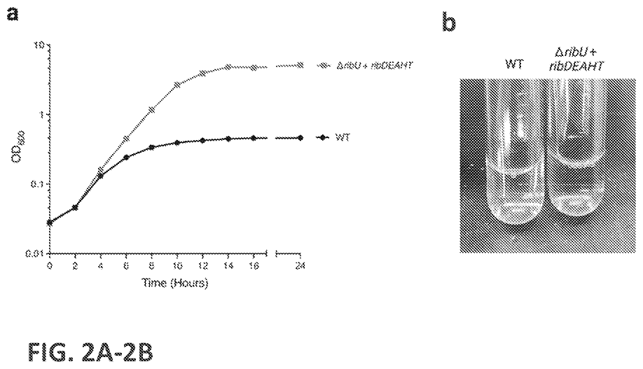

To determine if the virulence defect of the ΔribU strain was caused by riboflavin starvation in vivo, the ΔribU mutant was engineered to synthesize riboflavin by inserting the riboflavin operon ribDEAHT from the closely related Gram-positive bacterium B. subtilis onto the L. monocytogenes chromosome. This strain grew in colorless chemically defined synthetic media without riboflavin supplementation and turned the media yellow, the natural color of flavins (FIG. 2). Expression of the ribDEAHT operon rescued the ΔribU strain's virulence to wild-type L. monocytogenes levels in the spleens and livers of mice (FIG. 1c,d). Based on these observations it is concluded that RibU is essential for L. monocytogenes pathogenesis and that its function relates to flavin acquisition since de novo riboflavin production in the RibU-minus strain completely bypassed RibU's essentiality in vivo. L. monocytogenes uses RibU to grow in macrophages

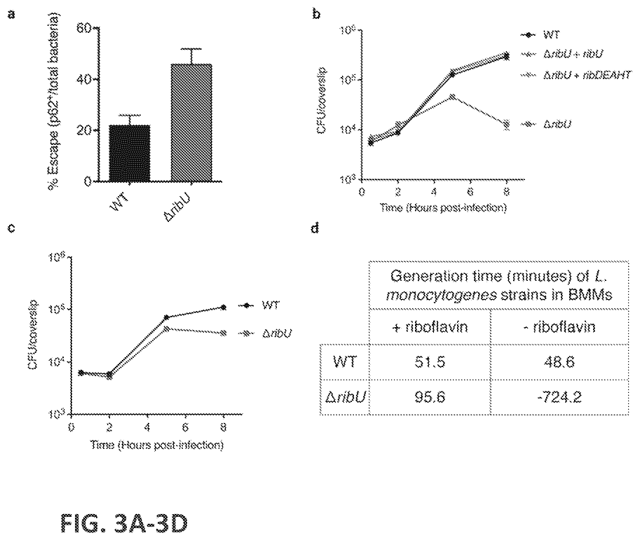

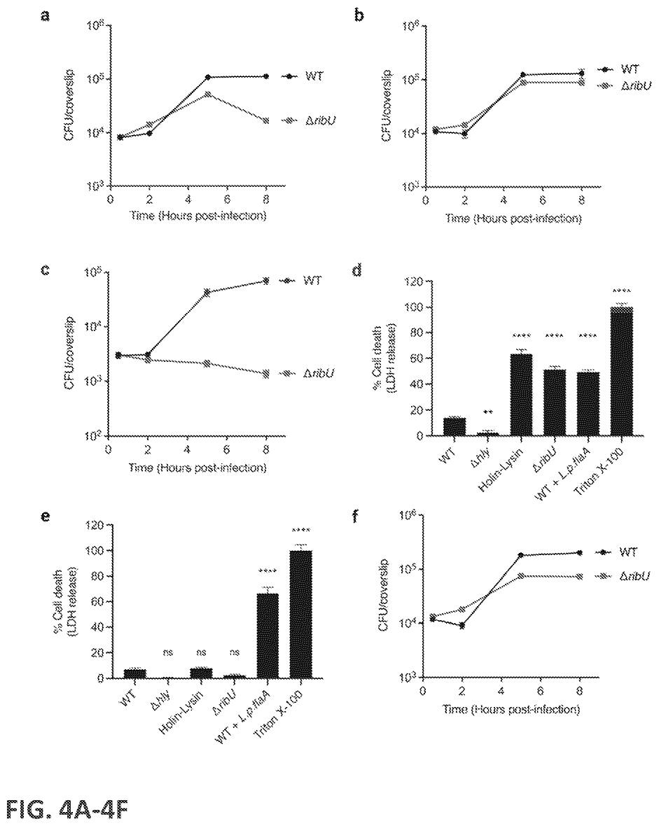

To study why RibU was essential for growth of L. monocytogenes in mice, infections were performed in vitro using bone marrow-derived macrophages (BMMs). At 2 hours post-infection, the ΔribU mutant had a small but significant growth advantage over wild-type L. monocytogenes (FIG. 4a), which was associated with an increase in phagosomal escape (FIG. 3a). However, during exponential growth (2-5 hours post-infection), the ΔribU mutant had an apparent defect in replication rate and showed a loss in CFUs during the late stages of infection (5-8 hours post-infection) (FIG. 4a). Complementation of the ΔribU strain with the ribU gene or the ribDEAHT operon completely restored the growth defects in BMMs (FIG. 3b).

To test if the ΔribU strain has an inherent intracellular virulence defect not related to riboflavin, BMMs were incubated with excess riboflavin (10 μM) prior to infection to increase the concentration of intracellular riboflavin. In this condition of riboflavin excess, the ΔribU mutant replicated to wild type levels (FIG. 4b). To assess if the growth of the ΔribU strain observed during the exponential growth phase in BMMs (FIG. 4a) is due to residual flavins from the medium, riboflavin-deficient BMMs were infected with riboflavin-starved bacteria. In this experiment, the ΔribU mutant and wild-type L. monocytogenes strains were incubated in chemically defined synthetic media lacking flavins for 16-18 hours prior to infection. The BMM cell culture media was replaced with media lacking riboflavin and the macrophages were incubated for 3 hours prior to infection with the riboflavin-starved bacteria. It was observed that riboflavin-starved wild-type L. monocytogenes were able to grow in riboflavin-deficient BMMs. However, the riboflavin-starved ΔribU mutant was unable to replicate in riboflavin depleted BMMs (FIG. 4c). The riboflavin-starved ΔribU mutant was able to grow in riboflavin-deficient BMMs supplemented with 1 M riboflavin just prior to infection (FIG. 3c).

To test if infection with the ΔribU strain led to host cell death, lactate dehydrogenase (LDH) release assays were performed. The results demonstrated that ΔribU caused a significant increase in host cell death (FIG. 4d). To test if ΔribU was triggering AIM2-dependent pyroptosis, LDH release assays were performed using AIM2 knockout (KO) BMMs and observed that the ΔribU mutant did not lead to LDH release (FIG. 4e). As a control, L. monocytogenes secreting flagellin (WT+L.p.flaA), which activates the NLRC4 inflammasome, still mediated cell death in infected AIM2 KO BMMs (FIG. 4c). The ΔribU mutant did not lead to cell death in AIM2 KO BMMs. Indeed, there was no loss in CFUs of the ΔribU mutant at 8 hours post infection (FIG. 4f). These data suggested that the RibU-minus strain lysed to some extent in vivo and activated AIM2-dependent pyroptosis, which negatively impacted the virulence of the strain.

L. monocytogenes Uses RibU to Scavenge FMN and FAD from the Cytosol of Host Cells

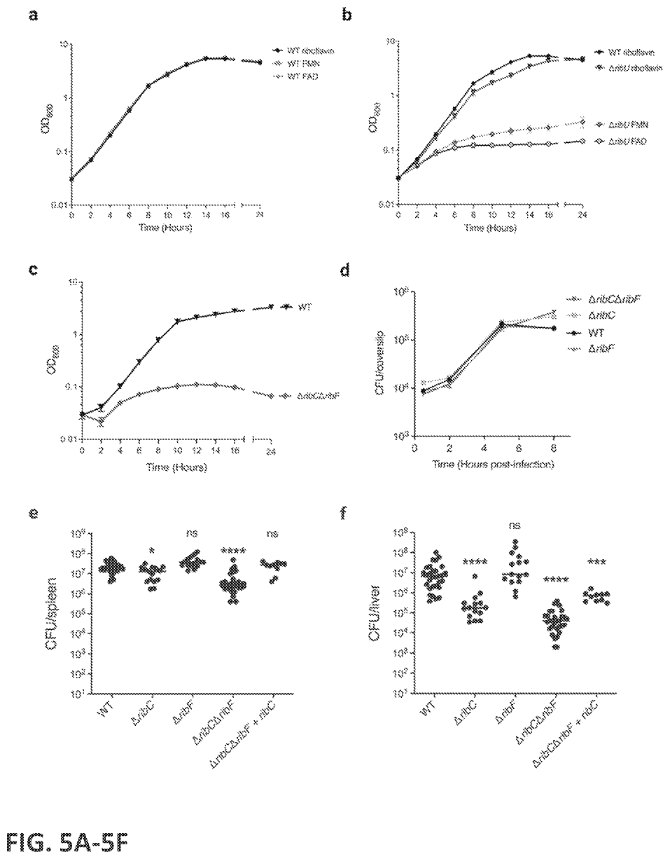

The doubling time of riboflavin-starved wild-type L. monocytogenes in riboflavin-deficient BMMs was very similar to the doubling time of wild-type L. monocytogenes growing in BMMs with riboflavin, 48.6 and 51.5 minutes, respectively (FIG. 3d). Both these results and the fact that mammalian cells rapidly convert riboflavin to FMN and FAD upon import indicates that wild-type L. monocytogenes imports FMN and/or FAD intracellularly to grow using RibU for their transport. To test if L. monocytogenes can import FMN and FAD to support growth, chemically defined synthetic media supplemented with riboflavin, FMN, or FAD as the sole flavin was used and it was found that wild-type L. monocytogenes grew in media containing each of the three flavins (FIG. 5a). By contrast, the ΔribU strain did not replicate in chemically defined media with FMN or FAD (FIG. 5b) and had only a slight defect in growth in media containing riboflavin. These results suggested that RibU is responsible for growth on FMN and FAD and that riboflavin can enter cells using RibU and/or another, yet to be identified, riboflavin transporter.

To test if L. monocytogenes utilizes RibU to scavenge FMN and FAD from the cytosol of host cells, an L. monocytogenes FMN and FAD auxotroph was generated by constructing strains lacking ribC, ribF, or both (ΔribCΔribF), enzymes responsible for converting riboflavin to FMN and FAD. Since FMN and FAD are essential cofactors, construction of this strain was performed in nutrient-rich media containing excess FMN and FAD to circumvent synthetic lethality. The ΔribCΔribF mutant was unable to replicate in chemically defined media with riboflavin as the sole flavin source (FIG. 5c).

If L. monocytogenes imports FMN and FAD from the host cytosol, the ΔribCΔribF mutant should not be impaired for intracellular growth. Indeed, these strains replicated intracellularly in BMMs to wild-type L. monocytogenes levels (FIG. 5d). To test if the ΔribC, ΔribF, and the ΔribCΔribF mutant L. monocytogenes strains grew in vivo, mouse virulence assays were performed. The ΔribC, ΔribF, and ΔribCΔribF mutants maintained their virulence and grew to high levels in both the spleens and livers of mice, albeit the ΔribC and ΔribCΔribF strains had statistically significant 2-log defects in the liver (FIG. 5e,f). Complementation of the ΔribCΔribF strain with the ribC gene with its endogenous promoter was able to restore most of the growth in the spleens and livers of infected mice (FIG. 5e,f). Thus, these results support a model in which L. monocytogenes uses RibU to import FMN and FAD from the cytosol of host cells.

The ΔribCΔribF Mutant Cannot Grow in Blood, Gallbladders, or the Gastrointestinal Tract

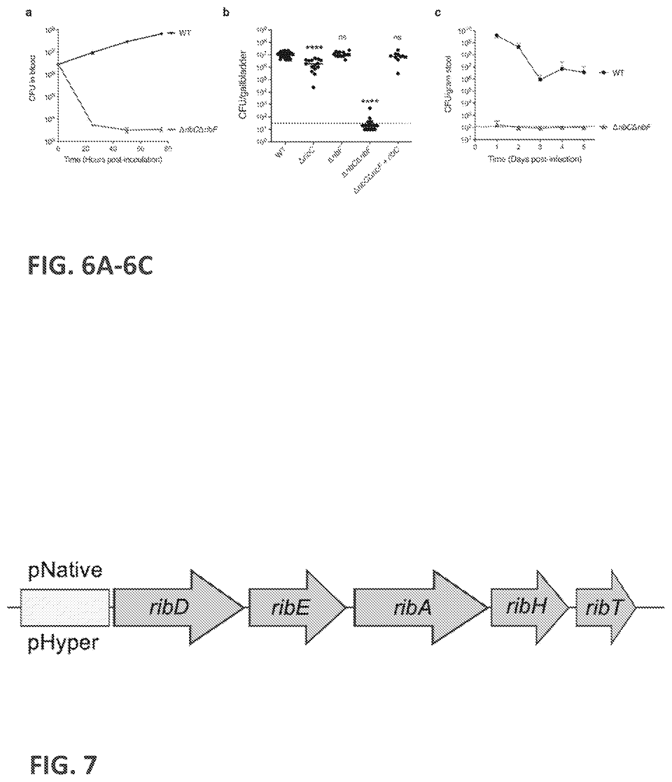

Wild-type L. monocytogenes can grow extracellularly in the gallbladder, blood, and the gastrointestinal (GI) tract of mice. During infection, L. monocytogenes colonizes the lumen of the gallbladder, which is connected to the liver through biliary ducts, and rapidly replicates extracellularly in the bile, establishing this organ as a bacterial reservoir. The ΔribCΔribF mutant was unable to colonize the gallbladder, while the ΔribC and ΔribF strains grew to wild-type L. monocytogenes levels (FIG. 6a). Complementation of the ΔribCΔribF mutant with a ribC gene with its endogenous promoter completely rescued the growth of the ΔribCΔribF strain in the gallbladder.

To assess if the ΔribCΔribF mutant strain can grow extracellularly in blood, a growth curve in defibrinated sheep's blood was performed and it was found that the ΔribCΔribF mutant did not replicate and observed a 2-3 logs CFU loss by 24 hours post-inoculation (FIG. 6b). To test if the ΔribCΔribF mutant can grow extracellularly in the lumen of the GI tract, mice were pre-treated for 2 days prior to the infection with streptomycin and were infected with 1×10⁸ CFU/mouse. Stool pellets were collected daily for 5 days and plated to assess bacterial burden. The ΔribCΔribF mutant had a 7-log defect in fecal pellet CFUs compared to wild-type L. monocytogenes at 24 hours post-infection (FIG. 6c). No CFUs were recovered from the ΔribCΔribF mutant infected mice following day 1 post-infection (FIG. 6c). Collectively, these observations suggest that the ΔribCΔribF strain cannot grow extracellularly in the gallbladder, blood, or the GI tract, and that this mutant is restricted to intracellular growth in vivo.

FIG. 1A illustrates the structures of riboflavin (black), FMN (red), and FAD (purple). FIG. 1B shows the broth growth curves of L. monocytogenes strains grown in nutrient-rich media. The means and standard deviations of three independent experiments are displayed. FIG. 1C-1D shows colony-forming units (CFUs) of L. monocytogenes strains in the spleens (C) and livers (D) of mice 48 hours after being infected with 1×10⁵ CFUs of the respective strains. The black lines represent the median CFUs for each strain. The dashed line represents the limit of detection.

FIG. 2A shows the broth growth curves of L. monocytogenes strains grown in chemically defined media lacking flavins. The means and standard deviations of three independent experiments are displayed. In chemically defined media lacking flavins, wild-type L. monocytogenes grows until it depletes its flavin pool. In contrast, the ΔribU+ribDEAHT strain grows to higher densities. FIG. 2B shows an image of the media supernatant of wild-type (left) and the ΔribU+ribDEAHT strain (right) after 24 hours of growth at 37° C. shaking.

FIG. 3A depicts the percentage of L. monocytogenes strains that colocalized with the autophagy receptor p62 in infected bone-marrow derived macrophages (BMMs). In BMMs treated with cytochalasin D, bacteria that escape phagosomes are tagged with p62. Percent phagosomal escape is calculated by counting the number of p62⁺ bacteria of total bacteria. The data show the means and standard errors of the mean of two independent experiments. FIG. 3B-3C shows intracellular growth curves of L. monocytogenes strains in BMMs. BMMs were infected at an MOI of 0.1 and CFUs were enumerated at the indicated times. (B) shows growth curves of indicated L. monocytogenes strains in wild-type BMMs. The data show the means and standard errors of the mean of two independent experiments. (C) shows growth curves of indicated flavin-starved L. monocytogenes strains in riboflavin- deprived wild-type BMMs (for 3 hours) supplemented with 1 μM riboflavin just prior to infection. The data represent the means and standard errors of the mean of three independent experiments. FIG. 3D illustrates the generation time of intracellularly growing L. monocytogenes strains in riboflavin sufficient and deprived BMMs between 2 to 5 hours. Negative values indicate that the number of recoverable bacteria was decreasing over time.

FIG. 4A-4C shows intracellular growth curves of L. monocytogenes strains in BMMs. BMMs were infected at an MOI of 0.1 and CFUs were enumerated at the indicated times. (A) shows growth curves of indicated L. monocytogenes strains in wild-type BMMs. The data show the means and standard errors of the mean of two independent experiments. (B) shows growth curves of indicated L. monocytogenes strains in wild-type BMMs incubated with cell culture media containing excess (10 μM) riboflavin during infection. The means and standard errors of the mean of three independent experiments are shown. (C) shows growth curves of indicated flavin starved L. monocytogenes strains in riboflavin-deficient wild-type BMMs. The data represent the means and standard errors of the mean of three independent experiments. FIG. 4D-E illustrates cell death of wild-type (D) or AIM2 KO (E) BMMs infected with specified L. monocytogenes strains. The data show the means and standard errors of the mean of three technical replicates from at least two and four independent experiments respectively. FIG. 4F shows intracellular growth curves of indicated L. monocytogenes strains in AIM2 KO BMMs. The means and standard errors of the mean of five independent experiments are shown.

FIG. 5A-5C shows the broth growth curves of L. monocytogenes strains grown in chemically defined synthetic media with different flavin sources. (A) shows growth curves of wild-type L. monocytogenes grown in media with riboflavin, FMN, or FAD as the only flavin source. The data show the means and standard deviations of four independent experiments. (B) shows growth curves of ΔribU mutant L. monocytogenes strain grown in media with riboflavin, FMN, or FAD as the sole flavin source. Wild-type L. monocytogenes grown in media with riboflavin is used as a reference. The data show the means and standard deviations of four independent experiments. (C) shows growth curves of indicated L. monocytogenes strains grown in media with riboflavin. The data show the means and standard deviations of two independent experiments. FIG. 5D shows intracellular growth curves of L. monocytogenes strains in murine BMMs. BMMs were infected at an MOI of 0.1 and CFUs were enumerated at the indicated times. The means and standard errors of the mean of two independent experiments are shown. FIG. 5E-5F shows the bacterial burdens in the spleens (5E) and livers (5F) of CD-1 mice infected intravenously with 1×10⁵ CFUs of indicated L. monocytogenes strains 48 hours after infection. The data show the combination of at least two independent experiments. The black lines represent the median CFUs for each strain.

FIG. 6A shows In vitro growth of L. monocytogenes strains in defibrinated sheep's blood. The means and standard errors of the mean of three independent experiments are shown. FIG. 6B shows the bacterial burdens in the gallbladder of CD-1 mice infected intravenously with 1×10⁵ CFUs of indicated L. monocytogenes strains 48 hours after infection. The data show the combination of at least two independent experiments. The black lines represent the median CFUs for each strain. The dashed line represents the limit of detection. FIG. 6C shows the bacterial burdens in the gastrointestinal tract of CD-1 mice infected orally with 1×10⁸ CFUs of indicated L. monocytogenes strains. Mice were pretreated orally with streptomycin prior to the infection. Stool samples were collected at days 1-5 and plated to determine the CFUs per gram of stool. The data show the means and standard deviations of the mean from a combination of three independent experiments. The dashed line represents the limit of detection.

### Example 2: MAIT Cell Activation Using Engineered L. monocytogenes Strains

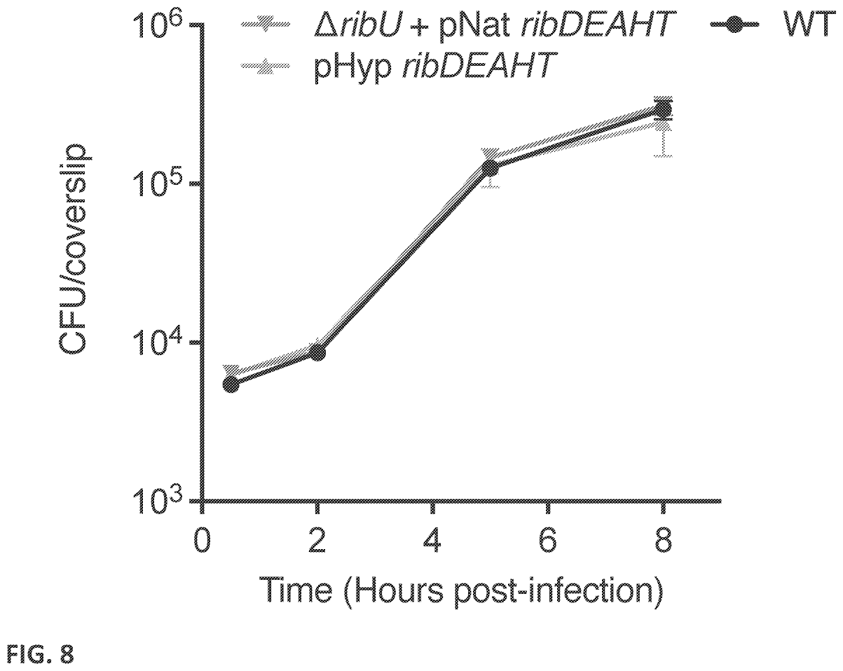

To address if L. monocytogenes is avoiding MAIT cells responses, L. monocytogenes strains that synthesize riboflavin de novo were engineered by cloning the riboflavin biosynthetic operon (ribDEAHT) from the closely related bacterium Bacillus subtilis into the L. monocytogenes chromosome. Two background strains were created with two different promoters, the first one is expressing the operon from a constitutively active promoter (pHyper) in the wild-type L. monocytogenes background—pHyper ribDEAHT (FIG. 7). The second strain expresses the riboflavin operon from the native B. subtilis promoter (pNative) (FIG. 7), an FMN riboswitch that turns on/off translation of ribDEAHT depending on the concentration of flavins inside the bacterium, in a riboflavin transporter (ribU)-minus L. monocytogenes background, which prevents the bacterium from importing flavins from the host cell and thus forces it to produce riboflavin de novo. To determine if the ribDEAHT strains were able to synthesize riboflavin de novo, a broth growth curve was performed in chemically defined media lacking flavins and showed that wild-type L. monocytogenes grows to some extent until it depletes the internal flavin pools, but that the ΔribU pNative ribDEAHT strain grew to higher densities (FIG. 2a). Furthermore, there is a visible change in color, from colorless to bright yellow (the natural color of flavins) in the media of the strain containing the ribDEAHT operon, suggesting that the ribDEAHT operon is functional and able to synthesize riboflavin de novo in the engineered strains. Since forcing L. monocytogenes to express an exogenous operon and synthesize riboflavin might impact its virulence or fitness, an intracellular growth curve in bone marrow-derived macrophages was performed. It was found that the strain expressing the ribDEAHT operon grew like wild-type L. monocytogenes intracellularly, thus producing riboflavin does not affect the intracellular fitness of L. monocytogenes in vitro (FIG. 3b and FIG. 8). These results suggested that the ribDEAHT strains were able to produce riboflavin and that they were not attenuated for intracellular growth.

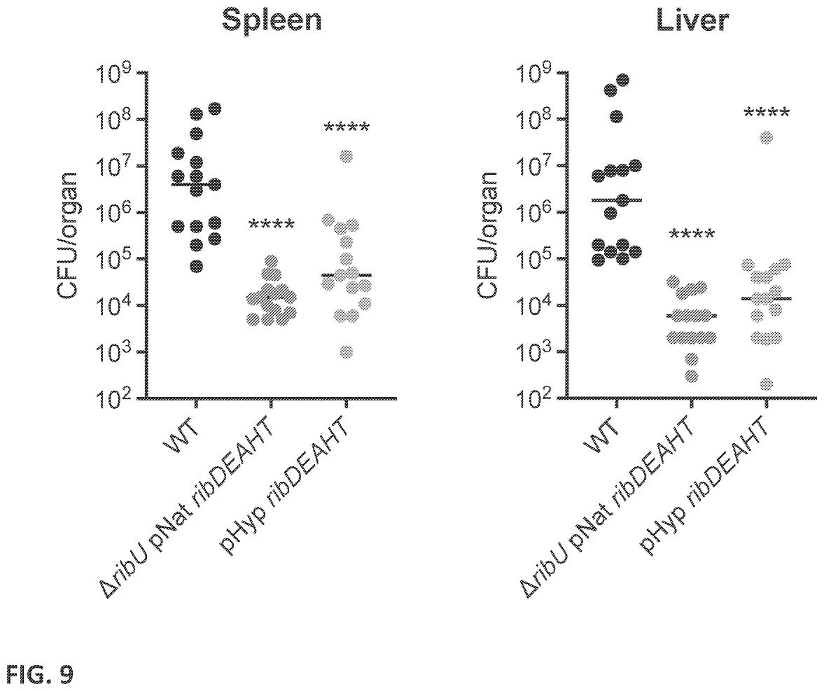

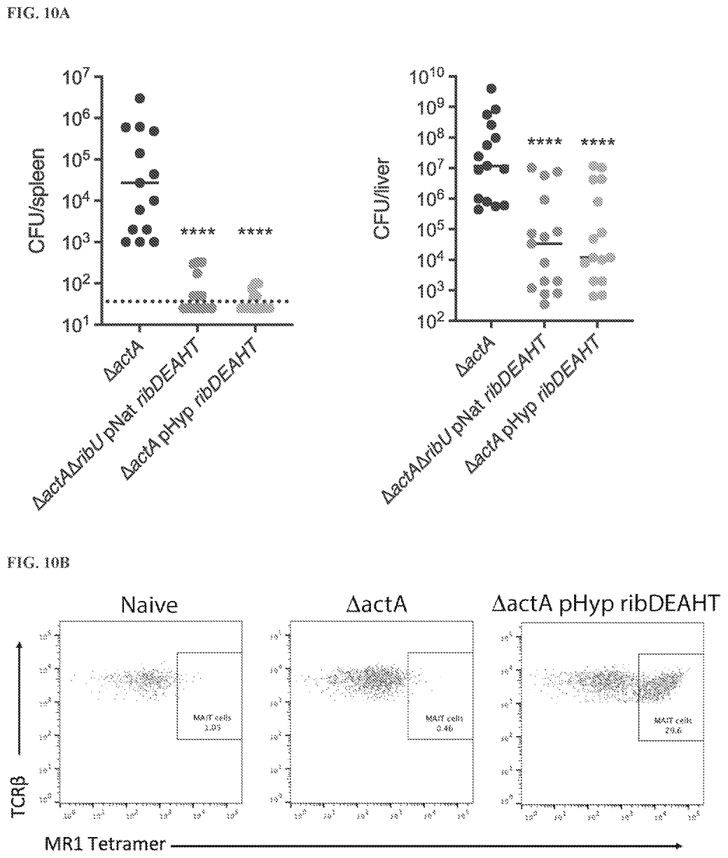

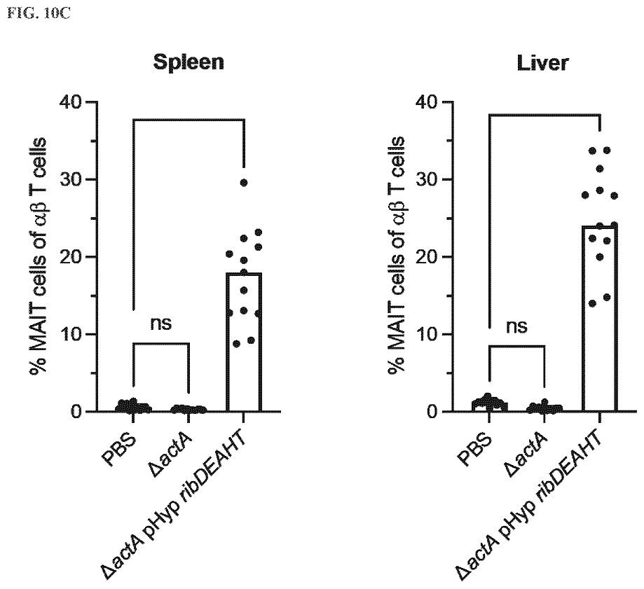

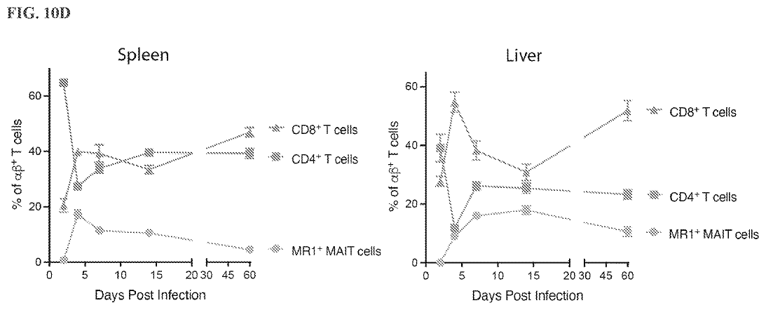

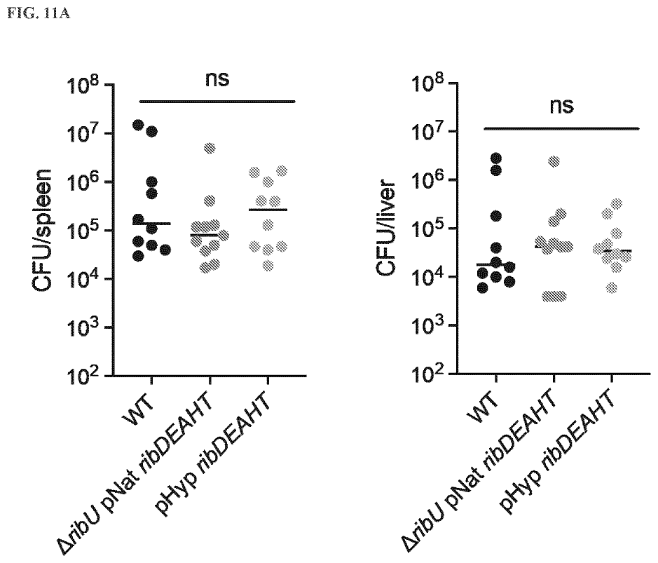

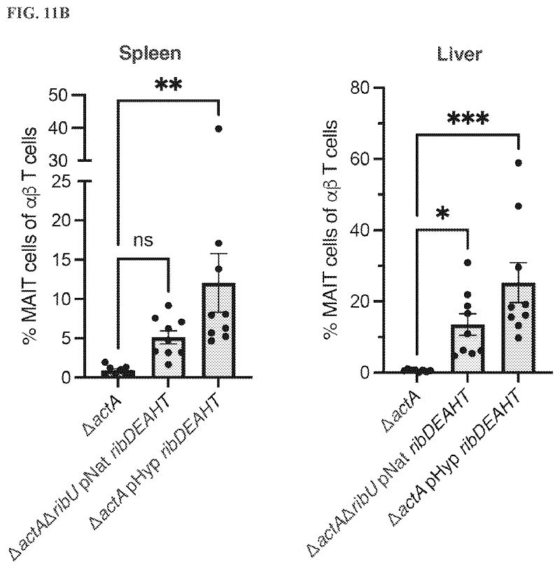

To determine if L. monocytogenes might be avoiding MAIT cell responses in vivo, mice were infected with wild-type L. monocytogenes or the riboflavin-producing (ribDEAHT) strains. It was found that by 4 days post-infection the pHyper ribDEAHT and ΔribU pNative ribDEAHT strains were highly attenuated in the spleens and livers of infected mice compared to wild-type L. monocytogenes, suggesting that MAIT cells are sensing the strains producing riboflavin and controlling their bacterial burden (FIG. 9). To determine if MAIT cells are responding to the riboflavin-producing strains and are getting activated and/or accumulating in infected tissues, mice were infected with the riboflavin-producing strains in the ΔactA background; the spleen and liver were collected to look at the number of MAIT cells (FIG. 10A). ActA is a virulence factor that allows L. monocytogenes to spread cell-to-cell and strains lacking this factor are less virulent, thus by using strains in this background mice can be infected with higher doses of bacteria allowing the chances of MAIT cells encountering cells infected with the ribDEAHT strains to increase. The frequencies of MAIT cells in perforin KO mice infected with ΔactA pHyper ribDEAHT were approximately 15% and 20% of all ab-T cells in the spleens and livers, respectively (FIG. 11B), similar to the frequencies observed in wild-type infected mice (FIG. 10C).

FIG. 7 is a diagram of the riboflavin operon, from B. subtilis, introduced into the L. monocytogenes chromosome with both pHyper (constitutive promoter) and pNative (inducible, native promoter from B. subtilis) depicted.

FIG. 8 shows intracellular growth curves of L. monocytogenes strains in murine BMMs. BMMs were infected at an MOI of 0.1 and CFUs were enumerated at the indicated times. The means and standard errors of the mean of two independent experiments are shown.

FIG. 9 shows the bacterial burdens in the spleens and livers of B6 mice infected intravenously with 1×10³ CFUs of indicated L. monocytogenes strains 96 hours after infection. The data show the combination of at least three independent experiments. The black lines represent the median CFUs for each strain.

### Example 3

Although a 5-fold increase of MAIT cells was observed in tissues infected with riboflavin-producing L. monocytogenes, the question was asked whether one can enhance this response by infecting mice with more bacteria. However, since the strains used in previous experiments were in the wild-type L. monocytogenes background, and the 50% lethal dose (LD₅₀) in mice is approximately 5-fold higher than the administered dose, infecting with higher doses would result in the death of the mice. Thus, it was decided to introduce the pNative and pHyper ribDEAHT operon constructs into the ActA-minus L. monocytogenes background (ΔactAΔribU pNative and pHyper ribDEAHT). ActA-minus L. monocytogenes cannot spread from cell to cell and thus is highly attenuated in mice (approximately 1000-fold compared to wild-type in BALB/C), which allowed infection with substantially more bacteria without killing the mice.

First, a virulence experiment was performed by infecting mice with a higher dose than the one previously administered (1×10⁷ CFU/mouse, instead of 1×10³ CFU/mouse) of the strains in the ActA-minus background to confirm that the attenuation observed with the riboflavin-producing strains in the wild-type background was reproducible. Indeed, at 4 days post-infection, the ActA-minus riboflavin-producing strains were 2-logs attenuated in the spleens and livers (FIG. 10A), as observed with the strains in the wild-type L. monocytogenes background. Strikingly, it was observed that the frequency of MAIT cells (CD3⁺TCR-β⁺MR1:5-OP-RU tetramer⁺T cells) in the mice infected with the ActA-minus pHyper ribDEAHT strain represented as high as 30% of αβ-T cells in the livers of infected mice (FIG. 10B). The median frequencies of MAIT cells in the spleens and livers of mice infected with the ActA-minus pHyper ribDEAHT strain were 15% and 20% of all αβ T cells, respectively (FIG. 10C). In contrast, the frequencies of MAIT cells in the spleens and livers of mice infected with the ΔactA L. monocytogenes control were between 0.5% and 1% of all T cells, which were the same as in naïve mice (FIG. 10C).

Subsequent to infection, MAIT cells can remain in tissues long after the infection has subsided. The question was asked whether that would be the case after infection with L. monocytogenes producing riboflavin. Mice were infected with ActA-minus pHyper ribDEAHT and collected the spleens and livers at 2, 4, 7, 14, and 60 days post-infection. From less than 1% of all αβ T cells at 2d post-infection, MAIT cell frequencies peaked at 20% in the spleens on day 4 post-infection, and in the livers on day 14 post-infection (FIG. 10D). At 60 days post-infection, MAIT cells comprised an average of 5% and 10% of all αβ T cells in the spleens and livers, respectively (FIG. 10D). CD8+ T cell frequencies started approximately at 20% in both organs and peaked 4 days post-infection at 40% in the spleens and 60% in the livers (FIG. 10D). In contrast, at day 2 post-infection, CD4+ T cells were at their highest frequencies at 60% in the spleens and 40% in the livers of αβ T cells (FIG. 10D). By day 4 post-infection, their frequencies were as low as 25% and 10% in the spleens and livers, respectively (FIG. 10D). At 60 days post-infection, CD4+ T cells ended with lower frequencies in both organs than their initial frequencies at the beginning of the experiments (FIG. 10D). These MAIT cell kinetic experiments showed that the frequencies of MAIT cells peaked at 4 days post-infection in the spleen and 14 days in the liver and were maintained for 60 days in these organs at higher frequencies than the frequencies of MAIT cells in naïve mice (FIG. 10C) and 2 day infected mice (FIG. 10D). These data suggest that MAIT cells strongly respond to attenuated riboflavin-producing L. monocytogenes strains by accumulating in high frequencies in infected organs and that they can remain in tissues at frequencies higher than those found in naïve mice.

FIG. 10A-10D. Infection with attenuated riboflavin-producing L. monocytogenes strains in ActA-minus backgrounds leads to substantial and sustained accumulation of MAIT cells in infected tissues. (A) Bacterial burdens in C57BL/6 mice infected intravenously with 1×10⁷ CFUs of indicated L. monocytogenes strains. At 4 d post-infection, the spleens (left) and livers (right) were harvested, homogenized, and plated to determine the CFUs per organ. The data show the combination of three independent experiments: ΔactA, ΔactAΔribU pNat ribDEAHT, and ΔactA pHyp ribDEAHT (n=15 mice). The black lines represent the median CFUs for each strain. The dashed line represents the limit of detection. Statistical significance of logarithmically transformed CFU values was determined using one-way ANOVA and Dunnett's posttest using WT as the control. ****P<0.0001. (B and C) (B) Representative flow cytometry plots from liver MAIT cells and (C) summarized data from spleens and livers showing the frequency of MAIT cells in naïve, ΔactA, or ΔactA pHyper ribDEAHT 4 d post-infection of C57BL/6 mice. Infection dose was 1×10⁷ CFU/mouse. MAIT cell percentages of live, CD45 positive, TCRβ positive, MR1:5-OP-RU tetramer positive T cells are shown. In (C), the means and SEMs of three independent experiments are shown: naive (n=14 mice), ΔactA (n=10 mice), and ΔactA pHyp ribDEAHT (n=13 mice). Statistical significance was determined using one-way ANOVA and Dunnett's posttest using (PBS) naïve mice as the control. ****P<0.0001; ns, not significant. P>0.05. (D) MAIT cell kinetic experiments showing the frequencies of MAIT cells, CD4⁺ T cells, and CD8⁺ T cells at 2, 4, 7, 14, and 60 d post-infection in mice infected with 1×10⁷ CFU/mouse of ΔactA pHyper ribDEAHT. Percentages of cells gated as live. CD45 positive, TCRβ positive, and then as MR1:5-OP-RU tetramer-positive CD4⁻/CD8⁻ negative cells (MAIT cells), CD4⁺/CD8⁻ (CD4⁺ T cells). and CD4⁻/CD8⁺ (CD8⁺ T cells), are shown. PBS; Phosphate buffer saline.

The data show that MAIT cells specifically restrict riboflavin-producing L. monocytogenes. The question was asked as to which mechanism(s) are involved. MAIT cells have two primary effector functions that mediate control of pathogens; production of cytokines which will activate bystander cells, or direct killing of infected cells using the cytolytic effectors granzyme B and perforin. Since L. monocytogenes is an intracellular pathogen. it was hypothesized that direct killing of infected cells would be the response employed by MAIT cells to restrict riboflavin-producing L. monocytogenes. Mice lacking perforin (perforin KO), which should prevent all cytotoxic cells including MAIT cells from directly killing infected cells, were infected. It was observed that the riboflavin-producing L. monocytogenes had no virulence defect compared to wild-type L. monocytogenes (FIG. 11A). To confirm that MAIT cells are still responding and accumulating in infected tissues, and not that the response at day 4 post-infection is different in perforin KO mice, these mice were infected with the ActA-minus riboflavin-producing L. monocytogenes strains and the frequencies of MAIT cells in organs were determined. The frequencies of MAIT cells in perforin KO mice were 15% and 20% of all αβ-T cells in the spleens and livers, respectively (FIG. 11B), similar to the frequencies observed in wild-type infected mice. These results suggested that the primary mechanism used by MAIT cells to restrict riboflavin-producing L. monocytogenes is direct killing of infected cells.

FIG. 11A-11B. Perforin-minus MAIT cells restrict riboflavin-producing L. monocytogenes. Bacterial burdens in perforin KO C57BL/6 mice, which lack the cytolytic effector perforin, infected intravenously with 1×10³ CFUs of indicated L. monocytogenes strains. At 4d post-infection, the spleens (left) and livers (right) were harvested, homogenized, and plated to determine the CFUs per organ. The data show the combination of two independent experiments: WT (n=10 mice), ΔribU pNat ribDEAHT (n=11 mice), and pHyper ribDEAHT (n=10 mice). Statistical significance of logarithmically transformed CFU values was determined using one-way ANOVA and Dunnett's posttest using WT as the control. ns, not significant, P>0.05. (B) Summarized data showing the frequency of MAIT cells in the spleens and livers of naïve, ΔactA, or ΔactA pHyper ribDEAHT at 4 d post-infection in perforin KO C57BL/6 mice. Infection dose was 1×10⁷ CFU/mouse. MAIT cell percentages of live, CD45 positive, TCRβ positive, MR1:5-OP-RU tetramer positive T cells are shown. The means and SEMs of two independent experiments are shown: ΔactA (n=8 mice), ΔactΔribU pNat ribDEAHT (n=9 mice), and ΔactA pHyp ribDEAHT (n=9 mice). Statistical significance was determined using one-way ANOVA and Dunnett's posttest using ΔactA mice as the control. ***P<0.001; **P<0.01; *P<0.05; ns, not significant, P>0.05.

### Example 4: RibC/RibF Strain with actA and inlB Mutations

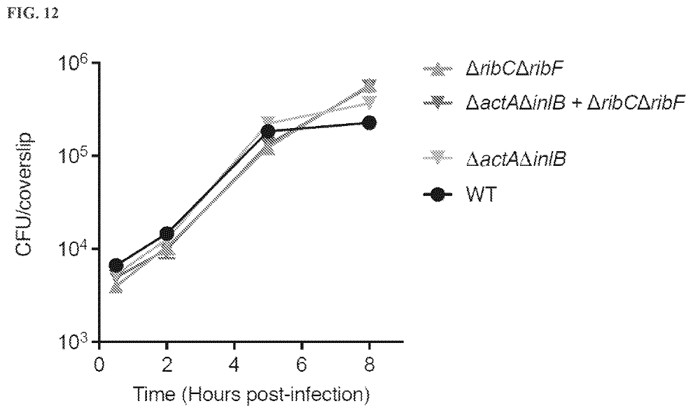

A ΔribC, ΔribF, ΔactA, ΔinIB strain of L. monocytogenes was constructed. This strain was compared to a ΔactA, ΔinIB strain of L. monocytogenes and to wild-type L. monocytogenes in macrophages in vitro. The data are shown in FIG. 12.

FIG. 12 shows intracellular growth curves of L. monocytogenes strains in wild-type murine BMMs. BMMs were infected at an MOI of 0.1 and CFUs were enumerated at the indicated times. The data shows that the ΔribCΔribFΔactAΔinIB strain of L. monocytogenes grew to wild-type, ΔactAΔinIB, and ΔribCΔribF strain levels intracellularly (no defects detected). Combining the actA and inlB deletions with the ΔribCΔribF strain of L. monocytogenes could make the ΔribCΔribF strain a safer vaccine vector or therapeutic, without affecting its intracellular replication.

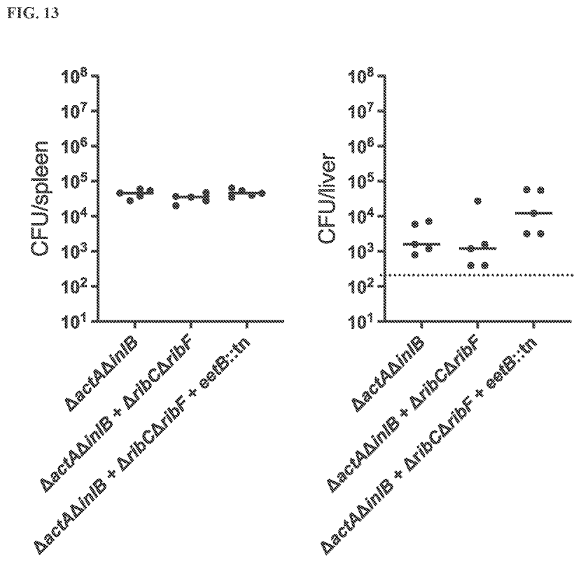

FIG. 13 depicts the bacterial burdens in the spleens and livers of CD-1 mice infected intravenously with 1×10⁵ CFUs of indicated L. monocytogenes strains 48 hours after infection. The data shows that the ΔribCΔribFΔactAΔinIB and ΔribCΔribFΔactAΔinlB+eetB::tn strains grow to ΔactAΔinIB CFU levels (no defects detected). Combining the actA and inlB deletions with the ΔribCΔribF strain, as well as disrupting the eetB gene (rendering the gene non-functional) in this background, led to strains that were as virulent as the control ΔactAΔinlB strain in vivo in mice.

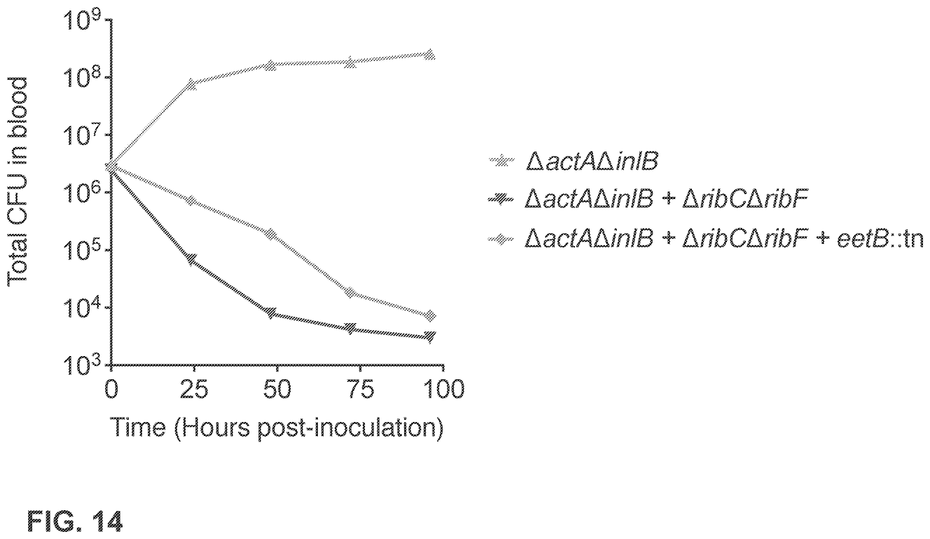

To assess if the ΔactAΔinlB+ΔribCΔribF and ΔactAΔinlB+ΔribCΔribF+eetB::tn mutant strains can grow extracellularly in blood, a growth curve in defibrinated sheep's blood was performed. The growth of the ΔactAΔinlB+ΔribCΔribF and ΔactAΔinlB+ΔribCΔribF+eetB::tn mutant strains of L. monocytogenes strains in blood was determined using defibrinated sheep's blood (HemoStat Laboratories, Dixon, CA, USA). The bacteria were grown to exponential phase for 2.5 hours, washed, and resuspended in 3 mL of defibrinated sheep's blood at a concentration of 1×10⁶/mL. Blood cultures were incubated at 37° C. shaking. The growth of L. monocytogenes in blood was monitored for 4 days by diluting the blood in 1X PBS and plating to determine the number of CFU in total blood. The data are shown in FIG. 14.

As shown in FIG. 14, it was found that at 24 hours after inoculation of blood, both ΔactAΔinlB+ΔribCΔribF and ΔactAΔinlB+ΔribCΔribF+eetB::tn did not grow and observed a 2-3 logs CFU loss for both strains, compared to ΔactAΔinlB which replicated to high numbers. The strains ΔactAΔinlB+ΔribCΔribF and ΔactAΔinlB+ΔribCΔribF+eetB::tn are highly attenuated in blood as was observed with the ΔribCΔribF mutant (FIG. 6A).

### Example 5: Vaccination

To test if the ΔribCΔribF mutant strain can robustly induce an adaptive immune response and generate protection against future L. monocytogenes challenge, mice were immunized with a 1×10⁵ dose of ΔactA and ΔactAΔribCΔribF. After allowing the mice to clear the infection and rest for 30 days, mice were infected with 5×10⁴ wild-type L. monocytogenes to examine the level of protection provided by the ΔactAΔribCΔribF strain. Generally, if a strain used to vaccinate can produce a robust immune response against L. monocytogenes, the mice will be able to mount a strong secondary immune response to the challenge and the mice will be protected. Different levels of protection can be observed depending on the effectiveness of the experimental strain at activating the adaptive immune response.

C57BL/6 mice were infected with a 1×10⁵ dose of exponentially growing ΔactA and ΔactAΔribCΔribF bacteria and mice were allowed to resolve the infection for 30 days. Then, mice were challenged (infected) with 5×10⁴ of exponentially growing wild-type L. monocytogenes to examine the level of protection provided by the ΔactAΔribCΔribF strain. Three days after challenge, spleens (left) and livers (right) were harvested and plated to count CFU per organ. ΔactA and ΔactAΔribCΔribF, n=10; PBS control (mice injected with 1X PBS as a control on day 0), n=5.

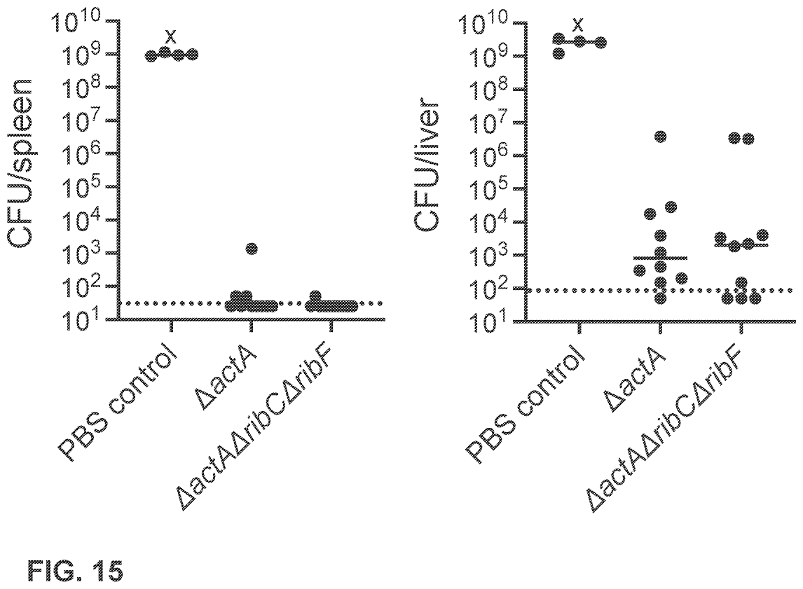

The data are shown in FIG. 15. As shown in FIG. 15, ΔactA and ΔactAΔribCΔribF provided the same level of protection suggesting that ΔactAΔribCΔribF can be a safe and effective vaccine platform or therapeutic for in vivo T cell/immune stimulation.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A variant Listeria bacterium comprising a mutation in gene required for flavin mononucleotide (FMN) and flavin adenine dinucleotide (FAD) biosynthesis, wherein the variant Listeria bacterium does not grow extracellularly in a mammal.

2. The variant Listeria bacterium of claim 1, wherein the gene required for FMN and FAD synthesis is a ribC gene and/or a ribF gene.

3. The variant Listeria bacterium of claim 1 or claim 2, wherein the variant Listeria bacterium is a conditionally obligate intracellular bacterium.

4. The variant Listeria bacterium of any one of claims 1-3, wherein the variant Listeria bacterium requires flavin mononucleotide and flavin adenine dinucleotide supplementation to grow.

5. The variant Listeria bacterium of any one of claims 2-4, wherein the mutation comprises a deletion of all or a portion of the ribC gene and/or the ribF gene.

6. The variant Listeria bacterium of any one of claim 5, wherein the variant is genetically modified to comprise a heterologous nucleic acid comprising a nucleotide sequence encoding at least one heterologous gene product.

7. The variant Listeria bacterium of claim 6, wherein the heterologous nucleic acid is integrated into the bacterial genome.

8. The variant Listeria bacterium of claim 6 or claim 7, wherein the at least one heterologous gene product comprises an antigen.

9. The variant Listeria bacterium of claim 8, wherein the antigen is a cancer-associated antigen.

10. The variant Listeria bacterium of any one of claims 1-9, wherein said variant Listeria bacterium is a variant Listeria monocytogenes bacterium.

11. The variant Listeria bacterium of any one of claims 1-10, wherein the variant Listeria bacterium further comprises one or more additional mutations that confers an attenuated phenotype on the bacterium and/or one or more additional mutations that provide a growth advantage.

12. The variant Listeria bacterium of claim 11, wherein the one or more additional mutations that confers an attenuated phenotype comprises a mutation in a gene selected from actA and inlB, and wherein the one or more additional mutations that provide a growth advantage comprises a mutation in an eetB gene.

13. A composition comprising:
a) a variant Listeria bacterium of any one of claims 1-12; and
b) a multispecific antibody.

14. The composition of claim 13, wherein the multispecific antibody comprises: i) a first antigen-binding site specific for a cancer-associated antigen; and ii) a second antigen-binding site specific for a T cell.

15. The composition of claim 14, wherein the T cell is a mucosal-associated invariant T (MAIT) cell, a γ/δ T cell, a CD8⁺ T cell, or a natural killer (NK) cell.

16. An immunogenic composition comprising the variant Listeria of any one of claims 1-12.

17. A method of inducing an immune response in an individual, the method comprising administering to the individual an effective amount of an immunogenic composition according to claim 16.

18. The method of claim 17, wherein the variant Listeria bacterium is genetically modified to comprise a heterologous nucleic acid comprising a nucleotide sequence encoding a heterologous polypeptide, and wherein said immune response is induced to the heterologous polypeptide.

19. The method of claim 18, wherein the heterologous polypeptide is an antigen.

20. The method of claim 18, wherein the antigen is a cancer-associated antigen.

21. The method of any one of claims 17-20, wherein said immune response comprises a gamma-delta T cell response.

22. A kit comprising a unit dose of the immunogenic composition of claim 16.

23. The kit of claim 22, wherein the unit dose is an oral dose.

24. The kit of claim 22, wherein the unit dose is injectable.

25. The kit of any one of claims 22-24, further comprising a recombinant expression vector comprising a nucleotide sequence encoding a heterologous antigen.

26. A 5-OP-RU precursor-producing variant Listeria that provides for production of the Mucosal-Associated Invariant T (MAIT) cell ligand 5-(2-oxopropylideneamino)-6-D-ribitylaminouracil (5-OP-RU).

27. The 5-OP-RU precursor-producing variant Listeria of claim 26, wherein the variant Listeria comprises a heterologous ribDEAHT operon, wherein the variant Listeria synthesizes riboflavin and stimulates proliferation of MAIT cells.

28. The 5-OP-RU precursor-producing variant Listeria of claim 26, wherein the variant Listeria comprises heterologous ribD and ribA genes.

29. The 5-OP-RU precursor-producing variant Listeria of any one of claims 26-28, wherein the variant is genetically modified to comprise a heterologous nucleic acid comprising a nucleotide sequence encoding at least one heterologous gene product.

30. The 5-OP-RU precursor-producing variant Listeria bacterium of claim 29, wherein the heterologous nucleic acid is integrated into the bacterial genome.

31. The 5-OP-RU precursor-producing variant Listeria bacterium of claim 29 or claim 30, wherein the at least one heterologous gene product comprises an antigen.

32. The variant Listeria bacterium of claim 31, wherein the antigen is a cancer-associated antigen.

33. The variant Listeria bacterium of claim 29 or claim 30, wherein the at least one heterologous gene product comprises a chimeric antigen receptor.

34. The variant Listeria bacterium of any one of claims 26-33, wherein the variant Listeria bacterium further comprises one or more additional mutations that confers an attenuated phenotype on the bacterium and/or one or more additional mutations that provide a growth advantage.

35. The variant Listeria bacterium of claim 34, wherein the one or more additional mutations that confers an attenuated phenotype comprises a mutation in a gene selected from actA and inlB, and wherein the one or more additional mutations that provide a growth advantage comprises a mutation in an eetB gene.

36. A composition comprising the variant Listeria of any one of claims 26-35.

37. A method of increasing the number and activation state of mucosal-associated invariant T cell (MAIT) cells in an individual, the method comprising administering to the individual an effective amount of the variant Listeria of any one of claims 26-35.

38. A method of treating cancer in an individual, the method comprising administering to the individual an effective amount of the variant Listeria of any one of claims 26-35.
